(19) United States
(12) Patent Application Publication
(10) Pub. No.: US 2004/0241637 A1
(43) Pub. Date: Dec. 2, 2004

(54) Method for establishing a standardized detection system for identifying a microorganism

(75) Inventor: Ching-Hsiang Hsu, Tainan County (TW)

Correspondence Address: BANNER & WITCOFF, 1001 G STREET N W, SUITE 1100, WASHINGTON, DC, 20001, US

(73) Assignee: GenMont Biotech Inc., Tainan County (TW)

(21) Appl. No.: 10/446,781

(22) Filed: May 29, 2003

(51) Int. Cl. C12Q 1/70; C12Q 1/68; G06F 19/00; G01N 33/48; G01N 33/50

(52) U.S. Cl. 435/5; 435/6; 702/20

(57) ABSTRACT

The present invention provides the tools and methodologies for establishing a standardized detection system for identifying a microorganism or a specific function in a microorganism by comparing the gene expression patterns in a test cell line.

### BACKGROUND OF THE INVENTION

1. Field of the Invention

The invention mainly relates to a method for establishing a standardized detection system for identifying a microorganism.

2. Description of the Related Art

With the development of molecular biology, many methods based on DNA or rDNA sequence analysis are developed for identification of a microorganism (Torriani et al., Differentiation of Lactobacillus plantarum, L. pentosus, and L. paraplantarum by recA Gene Sequence Analysis and Multiplex PCR Assay with recA Gene-Derived Primers. Appl Environ Microbiol. August 2001; 67(8): 3450-3454.). Examples of these methods are (1) randomly amplified polymorphic DNA (RAPD analysis) where a 10-15 bp primer participates for generating a fingerprint of the microorganism to be analyzed (Hayford et al., Use of Conserved Randomly Amplified Polymorphic DNA (RAPD) Fragments and RAPD Pattern for Characterization of Lactobacillus fermentum in Ghanaian Fermented Maize Dough. Appl Environ Microbiol. July 1999; 65(7): 3213-3221); (2) ribotyping through a Southern blot pattern formed on restriction enzymes digested fragments of chromosomal DNA of the microorganism to be identified; (3) pulsed-field electrophoresis (PFGE) providing a specific pattern of genomic DNA of the microorganism to be identified after electrophoresis; and (4) PCR analysis of 16S rRNA or 16S-23S rRNA intergenic spacer region sequence with species-specific primers (Tannock et al., Identification of Lactobacillus Isolates from the Gastrointestinal Tract, Silage, and Yoghurt by 16S-23S rRNA Gene Intergenic Spacer Region Sequence Comparisons. Appl Environ Microbiol. September 1999; 65(9): 4264-4267; Matsuki et al., Distribution of Bifidobacterial Species in Human Intestinal Microflora Examined with 16S rRNA-Gene-Targeted Species-Specific Primers. Appl Environ Microbiol. October 1999; 65(10): 4506-4512; Rossi et al., Genus- and Species-Specific PCR-Based Detection of Dairy Propionibacteria in Environmental Samples by Using Primers Targeted to the Genes Encoding 16S rRNA. Appl Environ Microbiol. September 1999; 65(9): 4241-4244; and Harmsen et al., Extensive Set of 16S rRNA-Based Probes for Detection of Bacteria in Human Feces. Appl Environ Microbiol. June 2002; 68(6): 2982-2990).

### SUMMARY OF THE INVENTION

The invention provides a method for establishing a standardized detection system for identifying a microorganism by comparing the gene expression patterns in a test cell line cultured with and without the microorganism.

One object of the invention is to provide a method for establishing a standardized detection system for identifying a microorganism, which comprises:

(i) providing a test cell line;

(ii) culturing the test cell line with and without the microorganism;

(iii) profiling the gene expression patterns of the test cell line cultured with and without the microorganism;

(iv) selecting a group of genes, whose expression in the test cell line cultured without the microorganism is significantly different from that cultured with the microorganism as markers; and

(v) storing the marker data obtained as the standardized detection system.

Another object of the invention is to provide a method for identifying an unknown microorganism as a standardized and recognized microorganism, which comprises:

(a) providing a standardized detection system for a standardized and recognized microorganism, wherein the detection system is prepared by the steps of:

(i) providing a test cell line;

(ii) culturing the test cell line with and without the standardized and recognized microorganism;

(iii) profiling the gene expression patterns of the test cell line cultured with and without the standardized and recognized microorganism;

(iv) selecting a group of genes, whose expression in the test cell line cultured without the standardized and recognized microorganism is significantly different from that cultured with the standardized and recognized microorganism as markers; and

(v) storing the marker data obtained as the standardized detection system;

(b) culturing the test cell line with and without the unknown microorganism;

(c) profiling the gene expression patterns of the test cell line cultured with and without the unknown microorganism;

(d) selecting a group of genes, whose expression in the test cell line cultured without the unknown microorganism is significantly different from that cultured with the unknown microorganism as markers;

(e) comparing the marker data obtained in step (d) with the marker data obtained in step (a), whereby the unknown microorganism is identified as the standardized and recognized microorganism if the marker data obtained in step (d) is similar to the marker data obtained in step (a) within an acceptable level.

The invention also provides a method for establishing a standardized detection system for detecting a specific function in a microorganism, which comprises:

(i) providing a test cell line;

(ii) culturing the test cell line with and without a reference microorganism having a specific function;

(iii) profiling the gene expression patterns related to the specific function of the test cell line cultured with and without the reference microorganism;

(iv) selecting a group of genes, whose expression in the test cell line cultured without the reference microorganism is significantly different from that cultured with the reference microorganism as markers; and

(v) storing the marker data obtained as the standardized detection system.

In another aspect, the invention provides a method for detecting a test microorganism having a specific function, which comprises:

(a) providing a standardized detection system for identifying the specific function in a reference microorganism, wherein the detection system is prepared by the steps of:

(i) providing a test cell line;

(ii) culturing the test cell line with and without the reference microorganism;

(iii) profiling the gene expression patterns related to the specific function of the test cell line cultured with and without the reference microorganism;

(iv) selecting a group of genes, whose expression in the test cell line cultured without the reference microorganism is significantly different from that cultured with the reference microorganism as markers; and

(v) storing the marker data obtained as the standardized detection system;

(b) culturing the test cell line with and without the test microorganism;

(c) profiling the gene expression pattern related to the specific function of the test cell line cultured with and without the test microorganism;

(d) selecting a group of genes, whose expression in the test cell line cultured without the test microorganism is significantly different from that with the test microorganism as markers;

(e) comparing the marker data obtained in step (d) with the marker data obtained in step (a), whereby the test microorganism is identified to have the specific function if the marker data obtained in step (d) is similar to the maker data obtained in step (a) within an acceptable level.

### DETAILED DESCRIPTION OF THE INVENTION

The present invention provides a method for establishing a standardized detection system for identifying a microorganism, which comprises:

(i) providing a test cell line;

(ii) culturing the test cell line with and without the microorganism;

(iii) profiling the gene expression patterns of the test cell line cultured with and without the microorganism;

(iv) selecting a group of genes, whose expression in the test to cell line cultured without the microorganism is significantly different from that cultured with the microorganism as markers; and

(v) storing the marker data obtained as the standardized detection system.

As used herein, the term “microorganism” refers to any genus and species of microorganism. In a preferred embodiment of the invention, the microorganism is a bacterium, and in a more preferred embodiment, the microorganism is a lactic acid bacterium.

The term “test cell line” as used herein refers to a cell line in which the gene expression patterns are profiled in different conditions. The cell line should be co-cultured with the microorganism to be identified. A test cell line group comprising more than one cell line can be used in the invention; wherein the cell lines should be derived from the same species. The use of the test cell line group minimizes cell variability for establishing the standardized detection system according to the invention. Preferably, the test cell line is selected from mammalian cells, and wherein more preferably, from human cells. The combination of the test cell line group depends on the microorganism to be identified. In some embodiments of the invention, the cell line includes liver cells such as Hep G2 cells, blood cells such as HL-60 and Turkat cells, and intestinal epithelium cells such as Caco-2 cells.

In the step (ii) of the method for establishing a standardized detection system for identifying a microorganism, the test cell line is cultured with and without the microorganism. The condition of each culture manipulation should be substantially equivalent or the same for minimizing experimental errors.

In the step (iii) of the method for establishing a standardized detection system for identifying a microorganism, the gene expression patterns of the test cell line cultured with and without the microorganism are profiled. The term “gene expression pattern” as used herein refers to a characteristic pattern of gene expression when culturing the test cell line in a given condition. The term “profiling the gene expression patterns” as used herein refers to a way of making unique patterns from the gene expression. The gene expression pattern can be used to distinguish a biological source from the other biological source. In one embodiment of the invention, the gene expression pattern is profiled by determining each gene expression in the test cell line. Normally, multiple assays for profiling are performed to obtain an average value of each gene expression.

Preferably, the gene expression patterns are conducted in a microarray. As used herein, the term “microarray” refers to a system for detecting multiple oligonucleotide interactions in multiple positions. The microarray is also known as terms “biochip”, “DNA chip” or “gene chip”. In one embodiment of the invention, the microarray comprises a chip including a solid support. The support comprises a plurality of spatially defined addresses and each said address has at least one copy of a single species of oligonucleotide probe attached thereto. When a sample comprising a target oligonucleotide having specific affinity for one of the probe, a specific signal can be detected and quantified by a scanner to give a recognition pattern.

In the step (iv) of the method for establishing a standardized detection system for identifying a microorganism, a group of genes are selected. The expression of the group of genes in the test cell line cultured without the microorganism is significantly different from that cultured with the microorganism, and therefore, the group of genes are used as markers. The gene expression pattern of the test cell line cultured without the microorganism acts as a baseline set of data by which various other sets of data are compared. The gene expression pattern of the test cell line cultured with the microorganism is compared with the baseline set of data. In one embodiment of the invention, the marker genes are selected to have an expression ratio of the test cell line cultured with the microorganism to that cultured without the microorganism larger than 0.2. Preferably, the ratio is larger than 1.5. Preferably, the expression difference between the marker genes of test, cell line cultured with the microorganism and that cultured without the microorganism is also incorporated into the markers. The difference indicates that the marker genes in the test cell line are up- or down-regulated in response to culturing with and without the microorganism.

According to the invention, the gene expression patterns when cultured with and without the microorganism in the test cell line are significantly different.

In the step (v) of the method for establishing a standardized detection system for identifying a microorganism, the marker data obtained in step (iv) is stored as the standardized detection system.

In one embodiment of the invention, the standardized detection system further comprises: (1) means for data acquisition and analysis; (2) means for database development; (3) means for integration and links; and (4) means for analysis of the resulting database. Preferably, the means are incorporated as a computer for storing the marker data according to the invention and for data analysis.

Another object of the invention is to provide a method for identifying an unknown microorganism as a standardized and recognized microorganism, which comprises:

(a) providing a standardized detection system for a standardized and recognized microorganism, wherein the detection system is prepared by the steps of:

(i) providing a test cell line;

(ii) culturing the test cell line with and without the standardized and recognized microorganism;

(iii) profiling the gene expression patterns of the test cell line cultured with and without the standardized and recognized microorganism;

(iv) selecting a group of genes, whose expression in the test cell line cultured without the standardized and recognized microorganism is significantly different from that cultured with the standardized and recognized microorganism as markers; and

(v) storing the marker data obtained as the standardized detection system;

(b) culturing the test cell line with and without the unknown microorganism;

(c) profiling the gene expression patterns of the test cell line cultured with and without the unknown microorganism;

(d) selecting a group of genes, whose expression in the test cell line cultured without the unknown microorganism is significantly different from that cultured with the unknown microorganism as markers;

(e) comparing the marker data obtained in step (d) with the marker data obtained in step (a), whereby the unknown microorganism is identified as the standardized and recognized microorganism if the marker data obtained in step (d) is similar to the marker data obtained in step (a) within an acceptable level.

As used herein, the term “unknown microorganism” refers to a microorganism to be identified. As used herein, the term “a standardized and recognized microorganism” refers to a microorganism which is well characterized and demonstrates a stable biological response in a particular biosystem. The standardized and recognized microorganism is usually standardized by genetical, chemical or biological tests well known by those skilled in this field and the identification criteria thereof have been set.

According to the invention, in the step (a), a standardized detection system for a standardized and recognized microorganism is provided and the detection system is prepared as mentioned above.

In the step (b), the unknown microorganism is co-cultured with and without the test cell line in the detection system. For the accuracy of identification, the culture conditions are substantially the same with the culture conditions in preparing the detection system.

In the step (c), the gene expression patterns of the test cell line cultured with and without the unknown microorganism are profiled. Preferably, a microarray can be used in preparing the detection system. Generally, multiple assays for the profiling are performed to obtain an average value of each gene expression. In one embodiment of the invention, the gene expression pattern of the test cell line cultured without the unknown microorganism is the gene expression pattern of the test cell line cultured without the standardized and recognized microorganism for preparing the detection system.

In the step (d) of the method for identifying an unknown microorganism as a standardized and recognized microorganism, a group of genes are selected as markers. The gene expression of the marker genes in the test cell line cultured without the unknown microorganism is significantly different from that cultured with the microorganism. The gene expression pattern of the test cell line cultured without the unknown microorganism acts as a baseline set of data. The gene expression pattern of the test cell line cultured with the unknown microorganism is compared with the baseline set of data.

In the step (e), a group of genes obtained in step (d) are selected as markers and compared with the marker data obtained in step (a), whereby the unknown microorganism is identified as the standardized and recognized microorganism if the marker data obtained in step (d) is similar to the marker data obtained in step (a) within an acceptable level. The difference of the gene expression can also be incorporated for comparison.

According to the invention, the detection system and the method for identifying an unknown microorganism can be applied in quality control when manufacturing a microorganism composition. The detection system provides a qualified and quantified index for checking or identifying the microorganism in the stocks or products.

The invention also provides a method for establishing a standardized detection system for detecting a specific function in a microorganism, which comprises:

(i) providing a test cell line;

(ii) culturing the test cell line with and without a reference microorganism having a specific function;

(iii) profiling the gene expression patterns related to the specific function of the test cell line cultured with and without the reference microorganism;

(iv) selecting a group of genes, whose expression in the test cell line cultured without the reference microorganism is significantly different from that cultured with the reference microorganism as markers; and

(v) storing the marker data obtained as the standardized detection system.

According to the invention, the method for establishing a standardized detection system for detecting a specific function in a microorganism, which is generally used for screening the microorganism having a specific function, is similar to the method for establishing a standardized detection system for identifying a microorganism as mentioned above.

As used herein, the term “a specific function” refers to a distinct biological effect peculiar to a microorganism on a given biosystem. The specific function includes, but is not limited to, an ability to changing physiological condition of a subject administrated with the microorganism or pharmaceutical effects of a disease or a syndrome.

As used herein, the term “reference microorganism” refers to a microorganism which is well characterized to have a specific function. The reference microorganism which is similar to the standardized and recognized microorganism as mentioned above is usually standardized by genetical, chemical or biological tests well known by those skilled in this field and the identifying criteria thereof have been set.

Particularly, in the step (iii) of the method for establishing a standardized detection system for detecting a specific function, in a microorganism, the gene expression patterns related to the specific function of the test cell line cultured with and without the reference microorganism are profiled. In one embodiment of the invention, the gene expression pattern is profiled by determining each gene expression found to be directly or indirectly related to the specific function in the test cell line. The genes found to be directly or indirectly related to the specific function include, but are not limited to, genes encoding proteins participating the machanism of the specific function, genes encoding regulatory factors of the production of these proteins, genes encoding proteins for transportion, or genes encoding cytokines for controlling the specific function.

In another aspect, the invention provides a method for detecting a test microorganism having a specific function, which comprises:

(a) providing a standardized detection system for identifying the specific function in a reference microorganism, wherein the detection system is prepared by the steps of:

(i) providing a test cell line;

(ii) culturing the test cell line with and without the reference microorganism;

(iii) profiling the gene expression patterns related to the specific function of the test cell line cultured with and without the reference microorganism;

(iv) selecting a group of genes, whose expression in the test cell line cultured without the reference microorganism is significantly different from that cultured with the reference microorganism as markers; and

(v) storing the marker data obtained as the standardized detection system;

(b) culturing the test cell line with and without the test microorganism;

(c) profiling the gene expression pattern related to the specific function of the test cell line cultured with and without the test microorganism;

(d) selecting a group of genes, whose expression in the test cell line cultured without the test microorganism is significantly different from that with the test microorganism as markers;

(e) comparing the marker data obtained in step (d) with the marker data obtained in step (a), whereby the test microorganism is identified to have the specific function if the marker data obtained in step (d) is similar to the maker data obtained in step (a) within an acceptable level.

According to the invention, the method for detecting a test microorganism having a specific function is similar to the method for identifying an unknown microorganism as a standardized and recognized microorganism as mentioned above. Particularly, as used herein, the term “test microorganism” refers to a microorganism to be screened to have the specific function.

The following Examples are given for the purpose of illustration only and are not intended to limit the scope of the present invention.

### EXAMPLE 1

### A Standardized Detection System for Identifying a Lactic Acid Bacterium

Test cell line and co-culturing with and without lactic acid bacteria: Hep G2 cell line was taken as the test cell line. The cells were refreshed by adding a fresh medium and cultured for 16 hours. The cells were divided into two groups, and wherein one was for the culture with the microorganism and the other was for the culture without the microorganism. When the cell concentration reached 1×10⁷/10 mL, 1×10⁷ lactic acid bacteria #7 (Lactobacillus plantarum), #73 (Lactobacillus gasseri) or #74 (Lactobacillus gasseri) were then added into the cells and co-cultured for 24 hours. The cells in the culture without the microorganism cells were cultured as well.

Profiling the gene expression patterns: The total RNAs were extracted from the test cell line cultured with and without the microorganism.

Eight μL of the RNA and 2 μL oligo poly-dT (12-18 mer, 0.1 μg/L) were well mixed and, kept at 70° C. for 10 minutes and then were cooled with ice for 2 minutes. The RNAs obtained were mixed with reverse transcription labeling mixture in dark and 3 μL Cy5-dUTP (1 mM), 2 μL SuperScript II (200 U/μL), and Rnasin (1 μL). The mixture was incubated at 42° C. for 2 hours for reverse transcription, and the reaction was terminated by adding 1.5 μL 20 mM EDTA. The RNAs were degraded by adding 1.5 μL 500 mM NaOH and heated for 10 minutes. The NaOH retained in the sample was then neutralized by adding 1.5 μL 500 mM HCl, and excess Cy5 was removed by spinning in ProbeQuant G-50 Micro Column. All the DNAs labeled with Cy5 were stored at −20° C.

Hundreds of genes chosen were amplified through polymerase chain reaction and then dissolved in spotting buffer for spotting on a chip. After denaturing at 95° C. for 3 minutes, the DNAs were attached to a glass carrier by ultra-violet rays using a spotting machine to form the chip for detection of gene expression.

The chip with the DNAs of chosen gene was pretreated with n-methyl-pyrilidinone/succinic anhydride/sodium borate and 5×SSC/0.1% SDS/1% BSA to eliminate nonspecific hybridization by blocking active groups on the glass carrier. The DNAs obtained from the test cell line labeled with Cy5 in hybridization buffer (50% formamide/0.2% SDS/10×SSC) were denatured at 95° C. for 5 minutes and cooled, and then loaded to the chip. Hybridizations were performed at 42° C. for 18 hours. Three solutions of 1×SSC/0.1% SDS, 0.1×SSC/0.1% SDS and 0.1×SSC were used to wash the samples and to remove the nucleotides which were not hybridized.

Gene expressions were detected and analyzed by scanning the chips using a fluorescence scanner and further quantified to obtain expression values. The fluorescent signals were quantified with GenePix™ Pro 3.0 (Axon Instruments, Inc.) and the backgrounds were then deduced, and then divided by the GAPDH (glyceraldehydes phosphate dehydrogenase, a house keeping gene). Mouse cDNA (ATBS) and plants DNA (RbCL) were both chosen as negative control.

Each of the expression values was represented in a mean of duplicate.

The gene expression profiles of the Hep G2 cell cultured with and without the lactic acid bacteria #7 (Lactobacillus plantarum), #73 (Lactobacillus gasseri), or #74 (Lactobacillus gasseri) were then obtained and listed in Table 1.

TABLE 1

| | | | Cultured | Cultured | Cultured |
|---|---|---|---|---|---|
| Gene No. | Gene name | Cell only | with #7 | with #73 | with #74 |
| TL00356 | Serine/threonine kinase 4 | 0.195 | 0.251 | 0.103 | 0.110 |
| TL00348 | Carnitine palmitoyltransferase I, | 0.195 | 0.257 | 0.090 | 0.102 |
| | liver | | | | |
| TL00340 | Branched chain alpha-ketoacid | 0.188 | 0.248 | 0.100 | 0.110 |
| | dehydrogenase kinase | | | | |
| TL00334 | Serine/threonine kinase 10 | 0.195 | 0.270 | 0.103 | 0.108 |
| TL00578 | FGF2 | 0.693 | 0.420 | 0.824 | 0.856 |
| TL00556 | Small inducible cytokine A4 | 0.195 | 0.238 | 0.105 | 0.121 |
| | (homologous to mouse Mip-1b) | | | | |
| TL00555 | C—C CHEMOKINE | 0.188 | 0.235 | 0.097 | 0.101 |
| | RECEPTOR TYPE 2 | | | | |
| TL00554 | Early growth response protein 1 | 0.201 | 0.266 | 0.115 | 0.114 |
| TL00553 | Small inducible cytokine A2 | 0.201 | 0.260 | 0.103 | 0.106 |
| | (monocyte chemotactic protein | | | | |
| | 1, | | | | |
| TL00552 | Interleukin 10 receptor | 0.396 | 0.323 | 0.258 | 0.104 |
| TL00551 | Transforming growth factor, | 0.218 | 0.273 | 0.139 | 0.120 |
| | beta receptor II (70-80 kD) | | | | |
| TL00549 | hepatocyte growth factor | 0.215 | 0.254 | 0.106 | 0.108 |
| | activator inh | | | | |
| TL00545 | Guanylate binding protein 1, | 0.201 | 0.229 | 0.092 | 0.106 |
| | interferon-inducible, 67 kD | | | | |
| TL00544 | interleukin-13 receptor mRNA, | 0.224 | 0.260 | 0.097 | 0.121 |
| | complete cds | | | | |
| TL00593 | IL-16 | 0.231 | 0.254 | 0.095 | 0.104 |
| TL00592 | TGFb receptor associate protein | 0.254 | 0.273 | 0.102 | 0.127 |
| | 1 | | | | |
| TL00591 | Transforming growth factor, | 0.231 | 0.241 | 0.097 | 0.108 |
| | beta 3 | | | | |
| TL00587 | EGF | 0.224 | 0.257 | 0.097 | 0.097 |
| TL00584 | Interleukin 4 receptor | 0.281 | 0.288 | 0.161 | 0.129 |
| TL00583 | Interleukin 7 | 0.208 | 0.273 | 0.098 | 0.118 |
| TL00582 | Lymphotoxin alpha (formerly | 0.356 | 0.301 | 0.203 | 0.150 |
| | tumor necrosis factor beta) | | | | |
| TL00581 | Annexin V (lipocortin V; | 0.508 | 0.539 | 0.476 | 0.243 |
| | endonexin II) | | | | |
| TL00579 | TGF-beta superfamily protein, | 0.373 | 0.408 | 0.361 | 0.254 |
| | complete | | | | |
| TL00578 | FGF2 | 1.086 | 1.022 | 1.039 | 0.958 |
| TL00438 | serine/threonine protein kinase | 0.221 | 0.357 | 0.160 | 0.127 |
| | KKIALRE | | | | |
| TL00437 | thymidine kinase 1, soluble | 0.323 | 0.345 | 0.211 | 0.260 |
| TL00428 | Homo sapiens protein kinase A | 0.218 | 0.301 | 0.132 | 0.178 |
| | binding protein AKAP110 | | | | |
| | mRNA, complete cds | | | | |
| TL00417 | MAP/ERK kinase kinase 3 | 0.756 | 1.179 | 0.776 | 0.956 |
| TL00594 | IL-1 receptor type 1 | 0.429 | 0.495 | 0.339 | 0.444 |
| TL00918 | IL-1β□ | 0.201 | 0.292 | 0.106 | 0.101 |
| TL00915 | INF-β | 0.201 | 0.304 | 0.115 | 0.102 |
| TL00610 | CD30 | 0.188 | 0.238 | 0.097 | 0.102 |
| TL00608 | HGF | 0.188 | 0.235 | 0.100 | 0.097 |
| TL00603 | IL-15 | 0.188 | 0.229 | 0.095 | 0.097 |
| TL00601 | IFN-γ | 0.201 | 0.245 | 0.102 | 0.104 |
| TL00598 | IL-5 receptor α | 0.198 | 0.260 | 0.113 | 0.114 |
| TL00575 | GAMMA-INTERFERON- | 0.505 | 0.470 | 0.373 | 0.332 |
| | INDUCIBLE PROTEIN IP-30 | | | | |
| | PRECURSOR | | | | |
| TL00574 | metallothionein I-B gene | 0.287 | 0.357 | 0.339 | 0.435 |
| TL00573 | INTERFERON-INDUCIBLE | 0.234 | 0.273 | 0.148 | 0.135 |
| | PROTEIN 9-27 | | | | |
| TL00571 | Interferon regulatory factor 5 | 0.238 | 0.273 | 0.103 | 0.099 |
| TL00564 | Fibroblast growth factor 7 | 0.231 | 0.226 | 0.100 | 0.095 |
| | (keratinocyte growth factor) | | | | |
| TL00563 | Interferon (alpha, beta and | 0.264 | 0.279 | 0.126 | 0.125 |
| | omega) receptor 2 | | | | |
| TL00561 | Interleukin 2 receptor gamma | 0.224 | 0.245 | 0.103 | 0.104 |
| | chain | | | | |
| TL00560 | INTERFERON-ALPHA/BETA | 0.205 | 0.226 | 0.094 | 0.099 |
| | RECEPTOR ALPHA CHAIN | | | | |
| | PRECURSOR | | | | |
| TL00559 | Interleukin 6 (B cell stimulatory | 0.205 | 0.251 | 0.100 | 0.101 |
| | factor 2) | | | | |
| TL00558 | Connective tissue growth factor | 0.221 | 0.273 | 0.161 | 0.190 |
| TL00557 | INTERFERON-GAMMA | 0.195 | 0.273 | 0.160 | 0.165 |
| | RECEPTOR ALPHA CHAIN | | | | |
| | PRECURSOR | | | | |
| TL00596 | metallothionein-III | 1.327 | 1.232 | 1.152 | 1.281 |
| TL00788 | Signal transducer and activator | 0.198 | 0.276 | 0.108 | 0.121 |
| | of transcription 3 (acute-ph | | | | |
| TL00775 | Angiopoietin-1 | 0.182 | 0.241 | 0.095 | 0.101 |
| TL00765 | Interleukin 6 signal transducer | 0.198 | 0.260 | 0.110 | 0.118 |
| | (gp130, oncostatin M receptor) | | | | |
| TL00762 | nitric oxide synthase 3 | 0.201 | 0.276 | 0.108 | 0.118 |
| | (endothelial cell) | | | | |
| TL00798 | Superoxide dismutase 2, | 0.185 | 0.248 | 0.100 | 0.104 |
| | mitochondrial | | | | |
| TL00668 | growth associated protein43 | 0.201 | 0.257 | 0.105 | 0.102 |
| TL00666 | stat2(Signal transducer and | 0.191 | 0.266 | 0.106 | 0.106 |
| | activator of transcription 2) | | | | |
| TL00655 | telomeric DNA sequence | 0.218 | 0.288 | 0.176 | 0.178 |
| TL00650 | p53-associated gene | 0.591 | 0.536 | 0.360 | 0.482 |
| TL00635 | Colony-stimulating factor 1 (M- | 0.188 | 0.229 | 0.100 | 0.099 |
| | CSF) | | | | |
| TL00632 | N-myc | 0.205 | 0.248 | 0.108 | 0.110 |
| TL00631 | c-src tyrosine kinase | 0.195 | 0.257 | 0.108 | 0.106 |
| TL00630 | myc proto-oncogene (c-myc) | 0.231 | 0.313 | 0.121 | 0.123 |
| TL00626 | Transforming growth factor, | 0.218 | 0.260 | 0.102 | 0.108 |
| | beta receptor III (betaglycan, 3 | | | | |
| TL00625 | stat-like protein (Fe65) mRNA, | 0.274 | 0.295 | 0.129 | 0.125 |
| | complete cds | | | | |
| TL00535 | Proteasome 26S subunit, | 0.254 | 0.285 | 0.116 | 0.110 |
| | ATPase, 3 | | | | |
| TL00534 | calpamodulin mRNA | 0.238 | 0.238 | 0.097 | 0.099 |
| TL00508 | Homo sapiens apoptotic | 0.208 | 0.219 | 0.095 | 0.099 |
| | protease activating factor 1 | | | | |
| | (Apaf-1) | | | | |
| TL00495 | UBIQUITIN CARBOXYL- | 0.244 | 0.238 | 0.103 | 0.102 |
| | TERMINAL HYDROLASE | | | | |
| | ISOZYME L1 | | | | |
| TL00484 | mitochondrial processing | 0.297 | 0.323 | 0.263 | 0.233 |
| | peptidase beta-subunit | | | | |
| TL00459 | protease inhibitor 12 (PI12; | 0.205 | 0.238 | 0.118 | 0.112 |
| | neuroserpin) | | | | |
| TL00458 | Ubiquitin-conjugating enzyme | 0.221 | 0.241 | 0.132 | 0.152 |
| | E2A (RAD6 homolog) | | | | |
| TL00457 | ubiquitin conjugating enzyme | 0.188 | 0.226 | 0.098 | 0.101 |
| | (UbcH8) mRNA, comp | | | | |
| TL00456 | Ubiquitin-conjugating enzyme | 0.221 | 0.295 | 0.495 | 0.457 |
| | E2B (RAD6 homolog) | | | | |
| TL01081 | Protein kinase clk1 | 0.191 | 0.266 | 0.103 | 0.101 |
| TL01071 | Interferon-gamma receptor alpha | 0.191 | 0.248 | 0.100 | 0.104 |
| | chain precursor | | | | |
| TL01068 | Beta-2 adrenergic receptor | 0.195 | 0.241 | 0.097 | 0.099 |
| TL01066 | apoptotic cysteine protease | 0.198 | 0.254 | 0.106 | 0.112 |
| | mch4 | | | | |
| TL00064 | RAC-ALPHA | 0.191 | 0.245 | 0.100 | 0.106 |
| | SERINE/THREONINE | | | | |
| | KINASE | | | | |
| TL00060 | Deoxyguanosine kinase | 0.356 | 0.345 | 0.266 | 0.249 |
| TL00059 | Choline kinase | 0.201 | 0.251 | 0.121 | 0.133 |
| TL00054 | NUCLEOSIDE | 0.426 | 0.502 | 0.216 | 0.214 |
| | DIPHOSPHATE KINASE A | | | | |
| TL00047 | Glycerol kinase | 0.393 | 0.339 | 0.202 | 0.235 |
| TL00046 | flavin containing | 0.188 | 0.238 | 0.100 | 0.104 |
| | monooxygenase 5 (FMO5) | | | | |
| | mRNA. | | | | |
| TL00036 | Hexokinase 1 | 0.449 | 0.458 | 0.410 | 0.178 |
| TL00031 | Adenosine kinase | 0.244 | 0.292 | 0.147 | 0.214 |
| TL00028 | Human 53K isoform of Type II | 1.106 | 0.950 | 0.253 | 0.231 |
| | phosphatidylinositol-4- | | | | |
| | phosphate 5-kinase (PIPK) | | | | |
| | mRNA, complete cds | | | | |
| TL00027 | Cyclin-dependent kinase 2 | 0.201 | 0.226 | 0.102 | 0.102 |
| TL00022 | TYROSINE-PROTEIN | 0.475 | 0.442 | 0.340 | 0.315 |
| | KINASE CSK | | | | |
| TL00018 | Pyruvate kinase, liver | 0.218 | 0.235 | 0.115 | 0.112 |
| TL00015 | CDC28 protein kinase 2 (CKS2) | 0.419 | 0.483 | 0.406 | 0.455 |
| | mRNA. | | | | |
| TL00900 | mitochondrial transcription | 0.264 | 0.298 | 0.103 | 0.101 |
| | termination factor | | | | |
| TL00894 | signal transducer and activator | 0.429 | 1.492 | 1.187 | 0.243 |
| | of transcription 6, interleukin-4 | | | | |
| | induced | | | | |
| TL00888 | Transcription factor AP-4 | 0.815 | 0.840 | 0.956 | 0.913 |
| | (activating enhancer-binding | | | | |
| | prote | | | | |
| TL00885 | STAT-1alpha/beta | 0.231 | 0.332 | 0.118 | 0.097 |
| TL00737 | c-rel | 0.195 | 0.248 | 0.111 | 0.104 |
| TL00873 | transcription elongation factor | 0.191 | 0.232 | 0.123 | 0.120 |
| | S-II, hS-II-T | | | | |
| TL00801 | DNA repair helicase ERCC3 | 0.627 | 0.580 | 0.539 | 0.609 |
| TL00570 | Bone morphogenetic protein | 0.191 | 0.260 | 0.102 | 0.104 |
| | receptor, type II (serine/threoni | | | | |
| TL00569 | ESTs, Highly similar to | 0.188 | 0.257 | 0.098 | 0.104 |
| | INTERFERON-INDUCED | | | | |
| | GUANYLATE-BINDIN | | | | |
| TL00568 | ESTs, Weakly similar to type 1 | 0.185 | 0.257 | 0.100 | 0.106 |
| | procollagen C-proteinase enha | | | | |
| TL00567 | Complement component 4- | 0.198 | 0.257 | 0.113 | 0.129 |
| | binding protein, alpha | | | | |
| TL00194 | Protein-tyrosine kinase 7 | 0.215 | 0.257 | 0.131 | 0.125 |
| TL00182 | TYROSINE-PROTEIN | 0.195 | 0.248 | 0.102 | 0.106 |
| | KINASE SYK | | | | |
| TL00177 | Human activated p21cdc42Hs | 0.191 | 0.254 | 0.111 | 0.114 |
| | kinase (ack) mRNA, complete | | | | |
| | cds | | | | |
| TL00165 | protein serine/threonine kinase | 0.198 | 0.235 | 0.108 | 0.112 |
| | stk2 | | | | |
| TL00161 | pyruvate dehydrogenase kinase | 0.191 | 0.238 | 0.102 | 0.112 |
| | isoenzyme 3 (PDK3) mRNA, | | | | |
| | complete cds | | | | |
| TL00159 | Deoxycytidine kinase | 0.195 | 0.270 | 0.103 | 0.106 |
| TL00150 | Lymphocyte-specific protein | 0.185 | 0.245 | 0.100 | 0.099 |
| | tyrosine kinase | | | | |
| TL00146 | Human putative serine/threonine | 0.185 | 0.241 | 0.097 | 0.101 |
| | protein kinase PRK (prk) | | | | |
| | mRNA, complete cds | | | | |
| TL00140 | Human integrin-linked kinase | 0.191 | 0.210 | 0.092 | 0.099 |
| | (ILK) mRNA, complete cds | | | | |
| TL00136 | stress-activated protein kinase 3 | 0.201 | 0.235 | 0.095 | 0.099 |
| | (SAPK3) mRNA. | | | | |
| TL00132 | Human diacylglycerol kinase | 0.218 | 0.251 | 0.121 | 0.110 |
| | zeta mRNA, complete cds | | | | |
| TL00119 | Urokinase-type plasminogen | 0.238 | 0.270 | 0.106 | 0.106 |
| | activator | | | | |
| TL00109 | RIBOSOMAL PROTEIN, S6 | 0.215 | 0.229 | 0.097 | 0.104 |
| | KINASE | | | | |
| TL00098 | Human adenylate kinase 2 | 0.274 | 0.357 | 0.145 | 0.118 |
| | (adk2) mRNA, complete cds | | | | |
| TL00096 | Protein kinase C, alpha | 0.248 | 0.245 | 0.105 | 0.099 |
| TL00084 | PROTO-ONCOGENE | 0.264 | 0.276 | 0.124 | 0.116 |
| | TYROSINE-PROTEIN | | | | |
| | KINASE FES/FPS | | | | |
| TL00082 | Human mitochondrial creatine | 0.231 | 0.310 | 0.126 | 0.095 |
| | kinase (CKMT) gene, complete | | | | |
| | cds | | | | |
| TL00079 | src-like kinase (slk) mRNA, | 0.201 | 0.219 | 0.111 | 0.114 |
| | complete cds. | | | | |
| TL00071 | Hemopoietic cell kinase | 0.182 | 0.213 | 0.092 | 0.093 |
| TL00070 | BMK1 alpha kinase mRNA, | 0.178 | 0.210 | 0.103 | 0.101 |
| | complete cds | | | | |
| TL00602 | PDGF associate protein | 0.191 | 0.248 | 0.106 | 0.114 |
| TL00599 | HGF activator | 0.191 | 0.238 | 0.108 | 0.104 |
| TL00597 | LIF | 0.422 | 0.398 | 0.316 | 0.330 |
| TL00595 | ICSBP1 | 0.195 | 0.254 | 0.100 | 0.099 |
| TL00672 | stat5a(Signal transducer and | 0.182 | 0.232 | 0.100 | 0.106 |
| | activator of transcription 5A) | | | | |
| TL00323 | MAP kinase-interacting | 0.191 | 0.282 | 0.116 | 0.104 |
| | serine/threonine kinase 1 | | | | |
| TL00656 | Tumor protein p53 | 0.195 | 0.238 | 0.126 | 0.123 |
| TL00319 | Protein kinase mitogen-activated | 0.198 | 0.248 | 0.134 | 0.127 |
| | 7 (MAP kinase) | | | | |
| TL00309 | Serine/threonine kinase 2 | 0.234 | 0.270 | 0.147 | 0.135 |
| TL00307 | Protein kinase mitogen-activated | 0.182 | 0.226 | 0.097 | 0.099 |
| | 8 (MAP kinase) | | | | |
| TL00302 | Fms-related tyrosine kinase 3 | 0.175 | 0.223 | 0.098 | 0.101 |
| TL00299 | Leukocyte tyrosine kinase | 0.185 | 0.223 | 0.095 | 0.101 |
| TL00290 | Mevalonate kinase | 0.185 | 0.204 | 0.094 | 0.104 |
| TL00287 | Human protein tyrosine kinase | 0.353 | 0.282 | 0.182 | 0.190 |
| | mRNA, complete cds | | | | |
| TL00280 | protein kinase, mitogen- | 0.188 | 0.216 | 0.092 | 0.101 |
| | activated 4 (MAP kinase 4; | | | | |
| | p63)(PRKM4) mRNA. | | | | |
| TL00278 | focal adhesion kinase (FAK) | 0.205 | 0.219 | 0.100 | 0.106 |
| | mRNA, complete cds | | | | |
| TL00276 | Creatine kinase B | 0.234 | 0.257 | 0.134 | 0.148 |
| TL00263 | Neurotrophic tyrosine kinase, | 0.221 | 0.229 | 0.103 | 0.101 |
| | receptor, type 3 (TrkC) | | | | |
| TL00244 | Glucokinase (hexokinase 4, | 0.218 | 0.229 | 0.100 | 0.102 |
| | maturity onset diabetes of the | | | | |
| | young 2) | | | | |
| TL00240 | H. sapiens mRNA for protein | 0.277 | 0.276 | 0.142 | 0.127 |
| | kinase CK1 | | | | |
| TL00239 | H. sapiens mRNA for FAST | 0.208 | 0.248 | 0.110 | 0.097 |
| | kinase | | | | |
| TL00236 | Human kinase (TTK) mRNA, | 0.211 | 0.263 | 0.126 | 0.108 |
| | complete cds | | | | |
| TL00233 | PHOSPHATIDYLINOSITOL | 0.208 | 0.219 | 0.135 | 0.125 |
| | 4-KINASE ALPHA | | | | |
| TL00231 | MAP kinase activated protein | 0.482 | 0.458 | 0.400 | 0.380 |
| | kinase | | | | |
| TL00414 | Cyclin-dependent kinase | 0.205 | 0.254 | 0.150 | 0.129 |
| | inhibitor 3 (CDK2-associated | | | | |
| | dual specificity phosphatase) | | | | |
| TL00405 | Cytokine suppressive anti- | 0.201 | 0.245 | 0.103 | 0.108 |
| | inflammatory drug binding | | | | |
| | protein 1 (p38 MAP kinase) | | | | |
| TL00402 | Phosphprylase kinase, beta | 0.198 | 0.248 | 0.106 | 0.110 |
| TL00392 | Tyrosine kinase 2 | 0.224 | 0.260 | 0.111 | 0.116 |
| TL00379 | pyruvate dehydrogenase kinase, | 0.251 | 0.248 | 0.118 | 0.118 |
| | isoenzyme 4 | | | | |
| TL00376 | phosphorylase kinase, alpha 2 | 0.221 | 0.226 | 0.105 | 0.104 |
| | (liver), glycogen storage disease | | | | |
| | IX | | | | |
| TL00373 | pyruvate dehydrogenase kinase, | 0.228 | 0.248 | 0.108 | 0.110 |
| | isoenzyme 3 | | | | |
| TL00371 | protein tyrosine kinase 6 | 0.234 | 0.235 | 0.111 | 0.102 |
| TL00370 | serine/threonine kinase 9 | 0.221 | 0.254 | 0.113 | 0.099 |
| TL00367 | Mitogen-activated protein | 0.205 | 0.257 | 0.118 | 0.101 |
| | kinase kinase kinase kinase 5 | | | | |
| TL00365 | serine/threonine protein-kinase | 0.201 | 0.245 | 0.119 | 0.118 |
| TL00360 | Pyridoxal (pyridoxine, vitamin | 0.175 | 0.226 | 0.108 | 0.101 |
| | B6) kinase | | | | |
| TL00761 | Tumor necrosis factor receptor 2 | 0.208 | 0.273 | 0.126 | 0.106 |
| TL00750 | IkB kinase beta subunit mRNA | 0.370 | 0.307 | 0.206 | 0.156 |
| TL00725 | Caspase-4 (Ich-2 protease | 0.238 | 0.273 | 0.140 | 0.125 |
| | precursor) | | | | |
| TL00723 | Caspase-9 (Human cysteine | 0.221 | 0.248 | 0.110 | 0.102 |
| | protease ice-lap6) | | | | |
| TL00717 | Phospholipase C | 0.492 | 0.498 | 0.318 | 0.351 |
| TL00715 | Caspase-8 (Apoptotic cysteine | 0.261 | 0.257 | 0.106 | 0.106 |
| | protease mch5 (mach-alpha-1)) | | | | |
| TL00707 | Bcl2, p53 binding protein | 0.248 | 0.248 | 0.123 | 0.112 |
| | Bbp/53BP2 (BBP/53BP2) | | | | |
| | mRNA, | | | | |
| TL00687 | Growth associated protein 43 | 0.241 | 0.235 | 0.115 | 0.101 |
| TL01032 | Glutathione S-transferase M1 | 0.304 | 0.292 | 0.255 | 0.214 |
| TL01020 | Protein kinase clk3 | 0.696 | 0.577 | 0.224 | 0.184 |
| TL00449 | serine/threonine kinase 11 | 0.198 | 0.241 | 0.123 | 0.116 |
| | (Peutz-Jeghers syndrome | | | | |
| TL00445 | syntaxin 8 | 0.409 | 0.329 | 0.261 | 0.218 |
| TL00633 | Breast cancer 1, early onset | 0.221 | 0.257 | 0.132 | 0.129 |
| | (BRCA1) mRNA | | | | |
| TL00274 | tyrosine kinase receptor (axl) | 0.205 | 0.248 | 0.108 | 0.114 |
| | mRNA, complete cds | | | | |
| TL00272 | JNK ACTIVATING KINASE 1 | 0.215 | 0.248 | 0.108 | 0.116 |
| TL01038 | Glutathione S-transferase 12 | 0.228 | 0.273 | 0.121 | 0.108 |
| | (microsomal) | | | | |
| TL01027 | Tumor necrosis factor-inducible | 0.238 | 0.254 | 0.111 | 0.101 |
| | protein TSG-6 precursor | | | | |
| TL01015 | Insulin-like growth factor I | 0.234 | 0.238 | 0.108 | 0.106 |
| | receptor precursor | | | | |
| TL00999 | Estrogen sulfotransferase (ste) | 0.228 | 0.226 | 0.106 | 0.106 |
| TL00998 | Monocyte chemotactic protein 1 | 0.248 | 0.245 | 0.118 | 0.099 |
| TL00991 | Leukocyte adhesion protein beta | 0.271 | 0.270 | 0.137 | 0.110 |
| | subunit | | | | |
| TL00977 | T-cell surface glycoprotein CD | 0.218 | 0.241 | 0.121 | 0.116 |
| | 4, p55 | | | | |
| TL00965 | voltage-gated calcium channel | 0.191 | 0.245 | 0.111 | 0.097 |
| | beta subunit | | | | |
| TL00793 | Topoisomerase (DNA) II beta | 0.182 | 0.219 | 0.110 | 0.108 |
| | (180 kD) | | | | |
| TL00705 | Cyclin-dependent kinase | 0.198 | 0.245 | 0.106 | 0.108 |
| | inhibitor 1C (p57, Kip2) | | | | |
| TL00662 | K-RAS | 0.208 | 0.245 | 0.118 | 0.114 |
| TL00653 | Rb | 0.201 | 0.251 | 0.111 | 0.102 |
| TL00641 | GTPase-activating protein ras | 0.231 | 0.251 | 0.134 | 0.129 |
| | p21 (RASA) | | | | |
| TL00606 | IGF binding protein 1 | 0.360 | 0.404 | 0.361 | 0.292 |
| TL00550 | IL-13Ra | 0.224 | 0.251 | 0.105 | 0.108 |
| TL00548 | vascular endothelial growth | 0.218 | 0.235 | 0.100 | 0.102 |
| | factor related protein vrp | | | | |
| | (VEGF-C) | | | | |
| TL00547 | transcriptional corepressor | 0.231 | 0.248 | 0.126 | 0.120 |
| | hKAP1/TIF1B mRNA, | | | | |
| | complete | | | | |
| TL00546 | fibroblast growth factor | 0.215 | 0.226 | 0.102 | 0.101 |
| | homologous factor 1 (FHF-1) m | | | | |
| TL00543 | FGF1 | 0.205 | 0.238 | 0.111 | 0.099 |
| TL00542 | vascular endothelial growth | 0.185 | 0.229 | 0.106 | 0.101 |
| | factor related protein VRP | | | | |
| TL00541 | Nerve growth factor receptor | 0.182 | 0.229 | 0.105 | 0.099 |
| TL00742 | bcl-xL mRNA. | 0.211 | 0.254 | 0.165 | 0.156 |
| TL00736 | Ros | 0.188 | 0.238 | 0.105 | 0.097 |
| TL00711 | Bcl-2 binding component 6 | 0.198 | 0.254 | 0.129 | 0.108 |
| | (bbc6) mRNA, complete cds. | | | | |
| TL00707 | Bcl2, p53 binding protein | 0.224 | 0.251 | 0.119 | 0.102 |
| | Bbp/53BP2 (BBP/53BP2) | | | | |
| | mRNA, | | | | |
| TL00589 | sis, PDGF B chain | 0.231 | 0.260 | 0.124 | 0.104 |
| TL00588 | Placental growth factor (P1GF) | 0.254 | 0.273 | 0.126 | 0.127 |
| TL00586 | Humig mRNA | 0.238 | 0.260 | 0.106 | 0.101 |
| TL00585 | CD27L RECEPTOR | 0.248 | 0.266 | 0.126 | 0.110 |
| | PRECURSOR | | | | |
| TL00580 | B-cell translocation gene 1, anti- | 0.238 | 0.238 | 0.124 | 0.108 |
| | proliferative | | | | |
| TL00577 | retinoic acid- and interferon- | 0.221 | 0.235 | 0.111 | 0.099 |
| | inducible 58K protein RI | | | | |
| TL00576 | EBI1-ligand chemokine, | 0.195 | 0.235 | 0.108 | 0.095 |
| | complete cds | | | | |
| TL00572 | eIF-1A, Y isoform (EIF1AY) | 0.215 | 0.254 | 0.152 | 0.192 |
| | mRNA, complete cds | | | | |
| | GAPDH | 1.000 | 1.000 | 1.000 | 1.000 |
| | b-actin | 0.333 | 0.455 | 0.340 | 0.389 |
| TL00751 | Caspase-1 | 0.195 | 0.241 | 0.106 | 0.101 |
| TL00566 | Homo sapiens F1F0-type ATP | 0.459 | 0.527 | 0.632 | 0.630 |
| | synthase subunit g mRNA, | | | | |
| | complete | | | | |
| TL00613 | NEU differentiation factor | 0.221 | 0.232 | 0.116 | 0.104 |
| TL00612 | IRF-1 | 0.218 | 0.235 | 0.108 | 0.099 |
| TL00611 | SDF-1b | 0.231 | 0.226 | 0.116 | 0.110 |
| TL00609 | ALK-1 | 0.215 | 0.235 | 0.118 | 0.104 |
| TL00607 | IL-12 bchain | 0.231 | 0.260 | 0.124 | 0.110 |
| TL00605 | EGR-1 | 0.188 | 0.238 | 0.124 | 0.104 |
| TL00604 | MGDF | 0.208 | 0.263 | 0.148 | 0.120 |

Selecting marker genes: A group of genes having the expression ratio in Hep G2 cultured with lactic acid bacteria #7, #73 or #74 to that cultured without the bacteria larger than 1.5 were selected. The results were shown in Table 2:

TABLE 2

| Gene No. | #7/cell | cell/#7 | Selected | #73/cell | cell/#73 | Selected | #74/cell | cell/#74 | Selected |
|---|---|---|---|---|---|---|---|---|---|
| TL00356 | 1.288 | 0.776 | | 0.530 | 1.886 | YES | 0.565 | 1.769 | YES |
| TL00348 | 1.32 | 0.758 | | 0.464 | 2.156 | YES | 0.526 | 1.900 | YES |
| TL00340 | 1.316 | 0.76 | | 0.532 | 1.881 | YES | 0.585 | 1.709 | YES |
| TL00334 | 1.385 | 0.722 | | 0.530 | 1.886 | YES | 0.555 | 1.800 | YES |
| TL00578 | 0.606 | 1.65 | YES | 1.189 | 0.841 | | 1.235 | 0.810 | |
| TL00556 | 1.224 | 0.817 | | 0.538 | 1.857 | YES | 0.624 | 1.603 | YES |
| TL00555 | 1.25 | 0.8 | | 0.514 | 1.944 | YES | 0.535 | 1.871 | YES |
| TL00554 | 1.324 | 0.756 | | 0.569 | 1.758 | YES | 0.566 | 1.768 | YES |
| TL00553 | 1.292 | 0.774 | | 0.513 | 1.950 | YES | 0.528 | 1.895 | YES |
| TL00552 | 0.815 | 1.227 | | 0.652 | 1.535 | YES | 0.264 | 3.795 | YES |
| TL00551 | 1.252 | 0.799 | | 0.637 | 1.570 | YES | 0.549 | 1.822 | YES |
| TL00549 | 1.184 | 0.845 | | 0.496 | 2.015 | YES | 0.504 | 1.983 | YES |
| TL00545 | 1.137 | 0.88 | | 0.457 | 2.190 | YES | 0.528 | 1.895 | YES |
| TL00544 | 1.159 | 0.863 | | 0.431 | 2.319 | YES | 0.541 | 1.848 | YES |
| TL00593 | 1.099 | 0.91 | | 0.412 | 2.428 | YES | 0.452 | 2.214 | YES |
| TL00592 | 1.073 | 0.932 | | 0.400 | 2.501 | YES | 0.500 | 1.999 | YES |
| TL00591 | 1.045 | 0.957 | | 0.419 | 2.387 | YES | 0.468 | 2.136 | YES |
| TL00587 | 1.145 | 0.873 | | 0.431 | 2.319 | YES | 0.431 | 2.319 | YES |
| TL00584 | 1.028 | 0.973 | | 0.575 | 1.739 | YES | 0.460 | 2.174 | YES |
| TL00583 | 1.312 | 0.762 | | 0.473 | 2.113 | YES | 0.566 | 1.767 | YES |
| TL00582 | 0.844 | 1.184 | | 0.570 | 1.754 | YES | 0.421 | 2.378 | YES |
| TL00581 | 1.061 | 0.943 | | 0.936 | 1.068 | | 0.478 | 2.093 | YES |
| TL00579 | 1.093 | 0.915 | | 0.969 | 1.032 | | 0.682 | 1.467 | |
| TL00578 | 0.941 | 1.062 | | 0.957 | 1.045 | | 0.883 | 1.133 | |
| TL00438 | 1.616 | 0.619 | YES | 0.722 | 1.385 | | 0.575 | 1.739 | YES |
| TL00437 | 1.066 | 0.938 | | 0.653 | 1.531 | YES | 0.804 | 1.244 | |
| TL00428 | 1.382 | 0.724 | | 0.607 | 1.647 | YES | 0.819 | 1.221 | |
| TL00417 | 1.56 | 0.641 | YES | 1.027 | 0.974 | | 1.265 | 0.790 | |
| TL00594 | 1.154 | 0.866 | | 0.789 | 1.267 | | 1.035 | 0.966 | |
| TL00918 | 1.448 | 0.691 | | 0.529 | 1.891 | YES | 0.500 | 2.002 | YES |
| TL00915 | 1.51 | 0.662 | YES | 0.569 | 1.758 | YES | 0.509 | 1.965 | YES |
| TL00610 | 1.266 | 0.79 | | 0.514 | 1.944 | YES | 0.545 | 1.836 | YES |
| TL00608 | 1.25 | 0.8 | | 0.532 | 1.881 | YES | 0.514 | 1.944 | YES |
| TL00603 | 1.216 | 0.822 | | 0.506 | 1.977 | YES | 0.514 | 1.944 | YES |
| TL00601 | 1.215 | 0.823 | | 0.505 | 1.981 | YES | 0.518 | 1.929 | YES |
| TL00598 | 1.314 | 0.761 | | 0.570 | 1.754 | YES | 0.575 | 1.739 | YES |
| TL00575 | 0.931 | 1.074 | | 0.738 | 1.355 | | 0.658 | 1.521 | YES |
| TL00574 | 1.245 | 0.803 | | 1.180 | 0.848 | | 1.513 | 0.661 | YES |
| TL00573 | 1.164 | 0.859 | | 0.633 | 1.579 | YES | 0.575 | 1.739 | YES |
| TL00571 | 1.148 | 0.871 | | 0.434 | 2.302 | YES | 0.415 | 2.408 | YES |
| TL00564 | 0.977 | 1.024 | | 0.433 | 2.310 | YES | 0.411 | 2.435 | YES |
| TL00563 | 1.057 | 0.946 | | 0.476 | 2.099 | YES | 0.474 | 2.108 | YES |
| TL00561 | 1.09 | 0.918 | | 0.460 | 2.174 | YES | 0.465 | 2.150 | YES |
| TL00560 | 1.103 | 0.907 | | 0.457 | 2.187 | YES | 0.482 | 2.074 | YES |
| TL00559 | 1.226 | 0.816 | | 0.489 | 2.046 | YES | 0.491 | 2.035 | YES |
| TL00558 | 1.233 | 0.811 | | 0.729 | 1.371 | | 0.858 | 1.165 | |
| TL00557 | 1.401 | 0.714 | | 0.820 | 1.219 | | 0.848 | 1.180 | |
| TL00596 | 0.929 | 1.077 | | 0.868 | 1.152 | | 0.965 | 1.036 | |
| TL00788 | 1.393 | 0.718 | | 0.546 | 1.832 | YES | 0.613 | 1.631 | YES |
| TL00775 | 1.33 | 0.752 | | 0.524 | 1.907 | YES | 0.554 | 1.805 | YES |
| TL00765 | 1.314 | 0.761 | | 0.554 | 1.805 | YES | 0.594 | 1.683 | YES |
| TL00762 | 1.37 | 0.73 | | 0.537 | 1.863 | YES | 0.584 | 1.711 | YES |
| TL00798 | 1.34 | 0.746 | | 0.541 | 1.848 | YES | 0.565 | 1.771 | YES |
| TL00668 | 1.277 | 0.783 | | 0.521 | 1.920 | YES | 0.509 | 1.965 | YES |
| TL00666 | 1.392 | 0.718 | | 0.556 | 1.798 | YES | 0.555 | 1.801 | YES |
| TL00655 | 1.324 | 0.755 | | 0.807 | 1.239 | | 0.819 | 1.221 | |
| TL00650 | 0.907 | 1.102 | | 0.609 | 1.642 | YES | 0.816 | 1.226 | |
| TL00635 | 1.216 | 0.822 | | 0.532 | 1.881 | YES | 0.525 | 1.907 | YES |
| TL00632 | 1.21 | 0.826 | | 0.528 | 1.894 | YES | 0.538 | 1.859 | YES |
| TL00631 | 1.32 | 0.758 | | 0.555 | 1.802 | YES | 0.546 | 1.832 | YES |
| TL00630 | 1.357 | 0.737 | | 0.524 | 1.910 | YES | 0.534 | 1.873 | YES |
| TL00626 | 1.194 | 0.837 | | 0.466 | 2.144 | YES | 0.497 | 2.014 | YES |
| TL00625 | 1.076 | 0.93 | | 0.471 | 2.123 | YES | 0.457 | 2.187 | YES |
| TL00535 | 1.123 | 0.891 | | 0.457 | 2.188 | YES | 0.433 | 2.309 | YES |
| TL00534 | 1.003 | 0.997 | | 0.407 | 2.455 | YES | 0.415 | 2.408 | YES |
| TL00508 | 1.055 | 0.948 | | 0.458 | 2.185 | YES | 0.475 | 2.107 | YES |
| TL00495 | 0.976 | 1.025 | | 0.423 | 2.366 | YES | 0.420 | 2.383 | YES |
| TL00484 | 1.087 | 0.92 | | 0.885 | 1.130 | | 0.786 | 1.273 | |
| TL00459 | 1.164 | 0.859 | | 0.575 | 1.738 | YES | 0.547 | 1.828 | YES |
| TL00458 | 1.092 | 0.916 | | 0.598 | 1.672 | YES | 0.687 | 1.457 | |
| TL00457 | 1.2 | 0.833 | | 0.523 | 1.912 | YES | 0.535 | 1.871 | YES |
| TL00456 | 1.333 | 0.75 | | 2.239 | 0.447 | YES | 2.068 | 0.484 | YES |
| TL01081 | 1.392 | 0.718 | | 0.539 | 1.854 | YES | 0.525 | 1.903 | YES |
| TL01071 | 1.294 | 0.773 | | 0.522 | 1.914 | YES | 0.545 | 1.834 | YES |
| TL01068 | 1.24 | 0.807 | | 0.497 | 2.012 | YES | 0.507 | 1.973 | YES |
| TL01066 | 1.282 | 0.78 | | 0.538 | 1.860 | YES | 0.565 | 1.769 | YES |
| TL00064 | 1.277 | 0.783 | | 0.522 | 1.914 | YES | 0.555 | 1.801 | YES |
| TL00060 | 0.967 | 1.034 | | 0.747 | 1.339 | | 0.697 | 1.434 | |
| TL00059 | 1.246 | 0.803 | | 0.601 | 1.664 | YES | 0.660 | 1.516 | YES |
| TL00054 | 1.178 | 0.849 | | 0.508 | 1.970 | YES | 0.504 | 1.986 | YES |
| TL00047 | 0.862 | 1.16 | | 0.513 | 1.948 | YES | 0.599 | 1.669 | YES |
| TL00046 | 1.266 | 0.79 | | 0.532 | 1.881 | YES | 0.555 | 1.803 | YES |
| TL00036 | 1.02 | 0.981 | | 0.913 | 1.096 | | 0.397 | 2.516 | YES |
| TL00031 | 1.194 | 0.838 | | 0.601 | 1.664 | YES | 0.878 | 1.139 | |
| TL00028 | 0.859 | 1.164 | | 0.229 | 4.366 | YES | 0.209 | 4.776 | YES |
| TL00027 | 1.121 | 0.892 | | 0.505 | 1.981 | YES | 0.509 | 1.965 | YES |
| TL00022 | 0.93 | 1.075 | | 0.716 | 1.396 | | 0.663 | 1.509 | YES |
| TL00018 | 1.079 | 0.926 | | 0.526 | 1.902 | YES | 0.514 | 1.946 | YES |
| TL00015 | 1.152 | 0.868 | | 0.970 | 1.031 | | 1.087 | 0.920 | |
| TL00900 | 1.128 | 0.887 | | 0.391 | 2.558 | YES | 0.381 | 2.625 | YES |
| TL00894 | 3.478 | 0.288 | YES | 2.767 | 0.361 | YES | 0.566 | 1.766 | YES |
| TL00888 | 1.031 | 0.97 | | 1.173 | 0.852 | | 1.120 | 0.893 | |
| TL00885 | 1.438 | 0.695 | | 0.510 | 1.962 | YES | 0.419 | 2.387 | YES |
| TL00737 | 1.272 | 0.786 | | 0.572 | 1.750 | YES | 0.536 | 1.866 | YES |
| TL00873 | 1.212 | 0.825 | | 0.640 | 1.562 | YES | 0.625 | 1.601 | YES |
| TL00801 | 0.925 | 1.081 | | 0.859 | 1.164 | | 0.971 | 1.029 | |
| TL00570 | 1.359 | 0.736 | | 0.531 | 1.884 | YES | 0.545 | 1.834 | YES |
| TL00569 | 1.366 | 0.732 | | 0.523 | 1.912 | YES | 0.555 | 1.803 | YES |
| TL00568 | 1.391 | 0.719 | | 0.541 | 1.848 | YES | 0.575 | 1.739 | YES |
| TL00567 | 1.298 | 0.77 | | 0.570 | 1.754 | YES | 0.652 | 1.535 | YES |
| TL00194 | 1.198 | 0.835 | | 0.609 | 1.642 | YES | 0.584 | 1.713 | YES |
| TL00182 | 1.272 | 0.786 | | 0.522 | 1.916 | YES | 0.546 | 1.832 | YES |
| TL00177 | 1.327 | 0.754 | | 0.581 | 1.720 | YES | 0.595 | 1.681 | YES |
| TL00165 | 1.187 | 0.842 | | 0.546 | 1.832 | YES | 0.565 | 1.769 | YES |
| TL00161 | 1.245 | 0.803 | | 0.531 | 1.884 | YES | 0.585 | 1.710 | YES |
| TL00159 | 1.385 | 0.722 | | 0.530 | 1.886 | YES | 0.546 | 1.832 | YES |
| TL00150 | 1.323 | 0.756 | | 0.541 | 1.848 | YES | 0.534 | 1.873 | YES |
| TL00146 | 1.306 | 0.766 | | 0.524 | 1.910 | YES | 0.544 | 1.838 | YES |
| TL00140 | 1.097 | 0.911 | | 0.480 | 2.082 | YES | 0.515 | 1.940 | YES |
| TL00136 | 1.168 | 0.856 | | 0.473 | 2.116 | YES | 0.490 | 2.040 | YES |
| TL00132 | 1.151 | 0.869 | | 0.555 | 1.801 | YES | 0.505 | 1.979 | YES |
| TL00119 | 1.135 | 0.881 | | 0.448 | 2.232 | YES | 0.447 | 2.236 | YES |
| TL00109 | 1.067 | 0.937 | | 0.451 | 2.217 | YES | 0.486 | 2.056 | YES |
| TL00098 | 1.305 | 0.767 | | 0.530 | 1.887 | YES | 0.429 | 2.328 | YES |
| TL00096 | 0.988 | 1.012 | | 0.424 | 2.361 | YES | 0.399 | 2.509 | YES |
| TL00084 | 1.045 | 0.957 | | 0.470 | 2.126 | YES | 0.438 | 2.281 | YES |
| TL00082 | 1.343 | 0.744 | | 0.545 | 1.836 | YES | 0.411 | 2.435 | YES |
| TL00079 | 1.09 | 0.917 | | 0.553 | 1.809 | YES | 0.566 | 1.768 | YES |
| TL00071 | 1.174 | 0.852 | | 0.506 | 1.974 | YES | 0.512 | 1.952 | YES |
| TL00070 | 1.179 | 0.849 | | 0.579 | 1.726 | YES | 0.564 | 1.772 | YES |
| TL00602 | 1.294 | 0.773 | | 0.556 | 1.798 | YES | 0.595 | 1.681 | YES |
| TL00599 | 1.245 | 0.803 | | 0.565 | 1.771 | YES | 0.545 | 1.834 | YES |
| TL00597 | 0.942 | 1.061 | | 0.748 | 1.336 | | 0.782 | 1.279 | |
| TL00595 | 1.304 | 0.767 | | 0.514 | 1.947 | YES | 0.507 | 1.973 | YES |
| TL00672 | 1.278 | 0.782 | | 0.551 | 1.815 | YES | 0.585 | 1.708 | YES |
| TL00323 | 1.474 | 0.678 | | 0.607 | 1.648 | YES | 0.545 | 1.834 | YES |
| TL00656 | 1.224 | 0.817 | | 0.646 | 1.548 | YES | 0.633 | 1.579 | YES |
| TL00319 | 1.251 | 0.8 | | 0.676 | 1.479 | | 0.642 | 1.558 | YES |
| TL00309 | 1.151 | 0.869 | | 0.626 | 1.596 | YES | 0.575 | 1.739 | YES |
| TL00307 | 1.243 | 0.804 | | 0.533 | 1.876 | YES | 0.544 | 1.840 | YES |
| TL00302 | 1.272 | 0.786 | | 0.562 | 1.778 | YES | 0.575 | 1.739 | YES |
| TL00299 | 1.204 | 0.83 | | 0.515 | 1.942 | YES | 0.544 | 1.838 | YES |
| TL00290 | 1.102 | 0.907 | | 0.506 | 1.976 | YES | 0.565 | 1.771 | YES |
| TL00287 | 0.799 | 1.252 | | 0.516 | 1.938 | YES | 0.537 | 1.861 | YES |
| TL00280 | 1.15 | 0.87 | | 0.489 | 2.046 | YES | 0.535 | 1.871 | YES |
| TL00278 | 1.072 | 0.932 | | 0.489 | 2.046 | YES | 0.519 | 1.926 | YES |
| TL00276 | 1.097 | 0.912 | | 0.571 | 1.750 | YES | 0.632 | 1.583 | YES |
| TL00263 | 1.035 | 0.966 | | 0.467 | 2.142 | YES | 0.455 | 2.199 | YES |
| TL00244 | 1.051 | 0.952 | | 0.459 | 2.178 | YES | 0.470 | 2.126 | YES |
| TL00240 | 0.995 | 1.005 | | 0.512 | 1.953 | YES | 0.459 | 2.181 | YES |
| TL00239 | 1.191 | 0.84 | | 0.527 | 1.896 | YES | 0.465 | 2.149 | YES |
| TL00236 | 1.247 | 0.802 | | 0.596 | 1.679 | YES | 0.512 | 1.953 | YES |
| TL00233 | 1.055 | 0.948 | | 0.652 | 1.535 | YES | 0.602 | 1.660 | YES |
| TL00231 | 0.95 | 1.053 | | 0.830 | 1.205 | | 0.788 | 1.270 | |
| TL00414 | 1.241 | 0.806 | | 0.733 | 1.364 | | 0.631 | 1.586 | YES |
| TL00405 | 1.215 | 0.823 | | 0.513 | 1.950 | YES | 0.537 | 1.861 | YES |
| TL00402 | 1.251 | 0.8 | | 0.538 | 1.860 | YES | 0.556 | 1.799 | YES |
| TL00392 | 1.159 | 0.863 | | 0.496 | 2.017 | YES | 0.516 | 1.939 | YES |
| TL00379 | 0.987 | 1.013 | | 0.469 | 2.130 | YES | 0.469 | 2.132 | YES |
| TL00376 | 1.021 | 0.98 | | 0.474 | 2.109 | YES | 0.472 | 2.119 | YES |
| TL00373 | 1.088 | 0.92 | | 0.475 | 2.107 | YES | 0.483 | 2.069 | YES |
| TL00371 | 1.003 | 0.997 | | 0.475 | 2.106 | YES | 0.437 | 2.287 | YES |
| TL00370 | 1.148 | 0.871 | | 0.511 | 1.959 | YES | 0.446 | 2.241 | YES |
| TL00367 | 1.256 | 0.796 | | 0.575 | 1.738 | YES | 0.491 | 2.035 | YES |
| TL00365 | 1.215 | 0.823 | | 0.593 | 1.687 | YES | 0.584 | 1.711 | YES |
| TL00360 | 1.29 | 0.775 | | 0.618 | 1.619 | YES | 0.575 | 1.739 | YES |
| TL00761 | 1.312 | 0.762 | | 0.605 | 1.653 | YES | 0.511 | 1.957 | YES |
| TL00750 | 0.831 | 1.203 | | 0.559 | 1.790 | YES | 0.421 | 2.376 | YES |
| TL00725 | 1.148 | 0.871 | | 0.591 | 1.693 | YES | 0.527 | 1.897 | YES |
| TL00723 | 1.12 | 0.893 | | 0.496 | 2.016 | YES | 0.463 | 2.158 | YES |
| TL00717 | 1.014 | 0.987 | | 0.646 | 1.548 | YES | 0.714 | 1.401 | |
| TL00715 | 0.986 | 1.014 | | 0.408 | 2.449 | YES | 0.408 | 2.454 | YES |
| TL00707 | 1.001 | 0.999 | | 0.495 | 2.019 | YES | 0.452 | 2.211 | YES |
| TL00687 | 0.976 | 1.025 | | 0.475 | 2.104 | YES | 0.417 | 2.396 | YES |
| TL01032 | 0.96 | 1.041 | | 0.839 | 1.191 | | 0.706 | 1.416 | |
| TL01020 | 0.828 | 1.207 | | 0.322 | 3.106 | YES | 0.264 | 3.783 | YES |
| TL00449 | 1.219 | 0.82 | | 0.619 | 1.615 | YES | 0.585 | 1.711 | YES |
| TL00445 | 0.804 | 1.243 | | 0.638 | 1.566 | YES | 0.533 | 1.875 | YES |
| TL00633 | 1.162 | 0.86 | | 0.598 | 1.672 | YES | 0.584 | 1.714 | YES |
| TL00274 | 1.21 | 0.826 | | 0.528 | 1.894 | YES | 0.556 | 1.797 | YES |
| TL00272 | 1.154 | 0.866 | | 0.504 | 1.985 | YES | 0.540 | 1.853 | YES |
| TL01038 | 1.198 | 0.835 | | 0.531 | 1.883 | YES | 0.475 | 2.105 | YES |
| TL01027 | 1.069 | 0.936 | | 0.468 | 2.135 | YES | 0.423 | 2.363 | YES |
| TL01015 | 1.017 | 0.984 | | 0.461 | 2.168 | YES | 0.453 | 2.205 | YES |
| TL00999 | 0.991 | 1.009 | | 0.467 | 2.139 | YES | 0.467 | 2.143 | YES |
| TL00998 | 0.988 | 1.012 | | 0.476 | 2.102 | YES | 0.399 | 2.509 | YES |
| TL00991 | 0.996 | 1.004 | | 0.507 | 1.974 | YES | 0.407 | 2.459 | YES |
| TL00977 | 1.108 | 0.902 | | 0.555 | 1.801 | YES | 0.531 | 1.882 | YES |
| TL00965 | 1.277 | 0.783 | | 0.581 | 1.720 | YES | 0.506 | 1.978 | YES |
| TL00793 | 1.209 | 0.827 | | 0.604 | 1.655 | YES | 0.596 | 1.678 | YES |
| TL00705 | 1.235 | 0.81 | | 0.538 | 1.860 | YES | 0.546 | 1.831 | YES |
| TL00662 | 1.176 | 0.85 | | 0.566 | 1.766 | YES | 0.548 | 1.826 | YES |
| TL00653 | 1.246 | 0.803 | | 0.553 | 1.809 | YES | 0.509 | 1.965 | YES |
| TL00641 | 1.086 | 0.921 | | 0.579 | 1.726 | YES | 0.559 | 1.790 | YES |
| TL00606 | 1.124 | 0.89 | | 1.004 | 0.996 | | 0.812 | 1.231 | |
| TL00550 | 1.117 | 0.895 | | 0.467 | 2.141 | YES | 0.482 | 2.075 | YES |
| TL00548 | 1.079 | 0.926 | | 0.459 | 2.178 | YES | 0.470 | 2.126 | YES |
| TL00547 | 1.072 | 0.933 | | 0.545 | 1.836 | YES | 0.517 | 1.933 | YES |
| TL00546 | 1.052 | 0.95 | | 0.474 | 2.111 | YES | 0.469 | 2.133 | YES |
| TL00543 | 1.164 | 0.859 | | 0.544 | 1.839 | YES | 0.482 | 2.074 | YES |
| TL00542 | 1.238 | 0.808 | | 0.576 | 1.736 | YES | 0.544 | 1.838 | YES |
| TL00541 | 1.261 | 0.793 | | 0.578 | 1.731 | YES | 0.544 | 1.840 | YES |
| TL00742 | 1.202 | 0.832 | | 0.779 | 1.284 | | 0.737 | 1.357 | |
| TL00736 | 1.266 | 0.79 | | 0.557 | 1.794 | YES | 0.514 | 1.944 | YES |
| TL00711 | 1.282 | 0.78 | | 0.652 | 1.535 | YES | 0.546 | 1.831 | YES |
| TL00707 | 1.117 | 0.895 | | 0.532 | 1.880 | YES | 0.457 | 2.190 | YES |
| TL00589 | 1.126 | 0.888 | | 0.538 | 1.860 | YES | 0.452 | 2.214 | YES |
| TL00588 | 1.073 | 0.932 | | 0.495 | 2.020 | YES | 0.500 | 1.999 | YES |
| TL00586 | 1.095 | 0.913 | | 0.448 | 2.232 | YES | 0.423 | 2.363 | YES |
| TL00585 | 1.076 | 0.929 | | 0.508 | 1.968 | YES | 0.445 | 2.249 | YES |
| TL00580 | 1.003 | 0.997 | | 0.523 | 1.913 | YES | 0.455 | 2.197 | YES |
| TL00577 | 1.063 | 0.941 | | 0.503 | 1.987 | YES | 0.446 | 2.241 | YES |
| TL00576 | 1.207 | 0.828 | | 0.555 | 1.802 | YES | 0.487 | 2.052 | YES |
| TL00572 | 1.184 | 0.845 | | 0.707 | 1.415 | | 0.893 | 1.119 | |
| | 1 | 1 | | 1.000 | 1.000 | | 1.000 | 1.000 | |
| | 1.364 | 0.733 | | 1.021 | 0.979 | | 1.167 | 0.857 | |
| TL00751 | 1.24 | 0.807 | | 0.547 | 1.829 | YES | 0.516 | 1.936 | YES |
| TL00566 | 1.148 | 0.871 | | 1.378 | 0.726 | | 1.373 | 0.728 | |
| TL00613 | 1.049 | 0.953 | | 0.525 | 1.904 | YES | 0.472 | 2.119 | YES |
| TL00612 | 1.079 | 0.926 | | 0.496 | 2.016 | YES | 0.453 | 2.208 | YES |
| TL00611 | 0.977 | 1.024 | | 0.503 | 1.989 | YES | 0.476 | 2.099 | YES |
| TL00609 | 1.096 | 0.912 | | 0.549 | 1.822 | YES | 0.486 | 2.056 | YES |
| TL00607 | 1.126 | 0.888 | | 0.538 | 1.860 | YES | 0.476 | 2.099 | YES |
| TL00605 | 1.266 | 0.79 | | 0.660 | 1.515 | YES | 0.555 | 1.803 | YES |
| TL00604 | 1.266 | 0.79 | | 0.714 | 1.401 | | 0.575 | 1.739 | YES |

The group of genes selected were used as markers. The differences between the expression of the marker genes cultured with and without the lactic acid bacteria can be incorporated as markers.

### EXAMPLE 2

### A Method for Identifying an Unknown Microorganism as a Standardized and Recognized Lactic Acid Bacteria #7, #73 and #74

Detection system: Detection systems to the standardized and recognized lactic acid bacteria #7, #73 and #74 were provided in Example 1.

The cultures of the test cell line with and without an unknown microorganism: The conditions of an unknown microorganism cultured with and without Hep G2 were similar to the conditions of the co-culture of Hep G2 with and without lactic acid bacteria #7, #73 or #74 as described in Example 1.

Profiling the gene expression patterns: The gene expression patterns of the cultures of Hep G2 with and without the unknown microorganism were obtained as described in Example 1. The results were shown in Table 3 as listed below.

Selecting marker genes: According to Table 3, a group of genes having an expression ratio in Hep G2 cultured with the unknown microorganism to that cultured without the unknown microorganism larger than 1.5 were selected. The results were shown in Table 3.

Comparing the marker data: The marker data were compared with the marker data of the lactic acid bacteria #7, #73, or #74 obtained in Example 1. The result were shown in Table 3.

TABLE 3

| | | Unknown/ | Cell/ | | | #73 | #74 |
|---|---|---|---|---|---|---|---|
| Gene No. | unknown | cell | unknown | Selected | #7 marker | marker | marker |
| TL00356 | 0.113 | 0.581 | 1.72 | YES | | YES | YES |
| TL00348 | 0.082 | 0.423 | 2.365 | YES | | YES | YES |
| TL00340 | 0.106 | 0.563 | 1.775 | YES | | YES | YES |
| TL00334 | 0.109 | 0.561 | 1.783 | YES | | YES | YES |
| TL00578 | 0.830 | 1.198 | 0.835 | | YES | | |
| TL00556 | 0.101 | 0.518 | 1.931 | YES | | YES | YES |
| TL00555 | 0.093 | 0.493 | 2.028 | YES | | YES | YES |
| TL00554 | 0.111 | 0.549 | 1.822 | YES | | YES | YES |
| TL00553 | 0.111 | 0.552 | 1.81 | YES | | YES | YES |
| TL00552 | 0.250 | 0.631 | 1.584 | YES | | YES | YES |
| TL00551 | 0.131 | 0.6 | 1.666 | YES | | YES | YES |
| TL00549 | 0.114 | 0.534 | 1.874 | YES | | YES | YES |
| TL00545 | 0.089 | 0.442 | 2.264 | YES | | YES | YES |
| TL00544 | 0.094 | 0.418 | 2.393 | YES | | YES | YES |
| TL00593 | 0.092 | 0.399 | 2.507 | YES | | YES | YES |
| TL00592 | 0.099 | 0.388 | 2.577 | YES | | YES | YES |
| TL00591 | 0.094 | 0.406 | 2.464 | YES | | YES | YES |
| TL00587 | 0.094 | 0.417 | 2.4 | YES | | YES | YES |
| TL00584 | 0.158 | 0.563 | 1.775 | YES | | YES | YES |
| TL00583 | 0.095 | 0.458 | 2.185 | YES | | YES | YES |
| TL00582 | 0.200 | 0.561 | 1.782 | YES | | YES | YES |
| TL00581 | 0.479 | 0.943 | 1.061 | | | | YES |
| TL00579 | 0.365 | 0.978 | 1.022 | | | | |
| TL00578 | 1.042 | 0.96 | 1.042 | | | | |
| TL00438 | 0.156 | 0.706 | 1.416 | | YES | | YES |
| TL00437 | 0.215 | 0.664 | 1.506 | YES | | YES | |
| TL00428 | 0.126 | 0.578 | 1.731 | YES | | YES | |
| TL00417 | 0.769 | 1.018 | 0.982 | | YES | | |
| TL00594 | 0.332 | 0.775 | 1.291 | | | | |
| TL00918 | 0.100 | 0.497 | 2.012 | YES | | YES | YES |
| TL00915 | 0.108 | 0.537 | 1.862 | YES | YES | YES | YES |
| TL00610 | 0.098 | 0.521 | 1.92 | YES | | YES | YES |
| TL00608 | 0.101 | 0.538 | 1.859 | YES | | YES | YES |
| TL00603 | 0.096 | 0.512 | 1.952 | YES | | YES | YES |
| TL00601 | 0.103 | 0.511 | 1.958 | YES | | YES | YES |
| TL00598 | 0.113 | 0.57 | 1.754 | YES | | YES | YES |
| TL00575 | 0.367 | 0.726 | 1.377 | | | | YES |
| TL00574 | 0.333 | 1.159 | 0.863 | | | | YES |
| TL00573 | 0.142 | 0.608 | 1.646 | YES | | YES | YES |
| TL00571 | 0.097 | 0.409 | 2.444 | YES | | YES | YES |
| TL00564 | 0.094 | 0.407 | 2.458 | YES | | YES | YES |
| TL00563 | 0.120 | 0.454 | 2.204 | YES | | YES | YES |
| TL00561 | 0.100 | 0.447 | 2.235 | YES | | YES | YES |
| TL00560 | 0.091 | 0.443 | 2.255 | YES | | YES | YES |
| TL00559 | 0.097 | 0.475 | 2.105 | YES | | YES | YES |
| TL00558 | 0.158 | 0.717 | 1.395 | | | | |
| TL00557 | 0.157 | 0.806 | 1.241 | | | | |
| TL00596 | 1.149 | 0.866 | 1.155 | | | | |
| TL00788 | 0.105 | 0.532 | 1.881 | YES | | YES | YES |
| TL00775 | 0.100 | 0.549 | 1.821 | YES | | YES | YES |
| TL00765 | 0.114 | 0.577 | 1.734 | YES | | YES | YES |
| TL00762 | 0.113 | 0.559 | 1.788 | YES | | YES | YES |
| TL00798 | 0.105 | 0.566 | 1.768 | YES | | YES | YES |
| TL00668 | 0.109 | 0.543 | 1.841 | YES | | YES | YES |
| TL00666 | 0.111 | 0.58 | 1.725 | YES | | YES | YES |
| TL00655 | 0.173 | 0.795 | 1.258 | | | | |
| TL00650 | 0.357 | 0.604 | 1.655 | YES | | YES | |
| TL00635 | 0.097 | 0.517 | 1.932 | YES | | YES | YES |
| TL00632 | 0.105 | 0.515 | 1.941 | YES | | YES | YES |
| TL00631 | 0.105 | 0.541 | 1.847 | YES | | YES | YES |
| TL00630 | 0.118 | 0.512 | 1.953 | YES | | YES | YES |
| TL00626 | 0.099 | 0.454 | 2.201 | YES | | YES | YES |
| TL00625 | 0.126 | 0.461 | 2.167 | YES | | YES | YES |
| TL00535 | 0.113 | 0.447 | 2.239 | YES | | YES | YES |
| TL00534 | 0.094 | 0.396 | 2.525 | YES | | YES | YES |
| TL00508 | 0.098 | 0.469 | 2.131 | YES | | YES | YES |
| TL00495 | 0.106 | 0.432 | 2.312 | YES | | YES | YES |
| TL00484 | 0.265 | 0.893 | 1.12 | | | | |
| TL00459 | 0.120 | 0.587 | 1.703 | YES | | YES | YES |
| TL00458 | 0.135 | 0.609 | 1.642 | YES | | YES | |
| TL00457 | 0.101 | 0.536 | 1.866 | YES | | YES | YES |
| TL00456 | 0.498 | 2.25 | 0.444 | YES | | YES | YES |
| TL01081 | 0.101 | 0.526 | 1.901 | YES | | YES | YES |
| TL01071 | 0.097 | 0.509 | 1.964 | YES | | YES | YES |
| TL01068 | 0.094 | 0.484 | 2.066 | YES | | YES | YES |
| TL01066 | 0.104 | 0.525 | 1.905 | YES | | YES | YES |
| TL00064 | 0.097 | 0.509 | 1.964 | YES | | YES | YES |
| TL00060 | 0.264 | 0.74 | 1.352 | | | | |
| TL00059 | 0.123 | 0.613 | 1.63 | YES | | YES | YES |
| TL00054 | 0.219 | 0.514 | 1.947 | YES | | YES | YES |
| TL00047 | 0.204 | 0.52 | 1.924 | YES | | YES | YES |
| TL00046 | 0.103 | 0.545 | 1.835 | YES | | YES | YES |
| TL00036 | 0.412 | 0.918 | 1.089 | | | | YES |
| TL00031 | 0.149 | 0.611 | 1.636 | YES | | YES | |
| TL00028 | 0.256 | 0.231 | 4.323 | YES | | YES | YES |
| TL00027 | 0.104 | 0.517 | 1.933 | YES | | YES | YES |
| TL00022 | 0.345 | 0.725 | 1.379 | | | | YES |
| TL00018 | 0.119 | 0.545 | 1.834 | YES | | YES | YES |
| TL00015 | 0.411 | 0.98 | 1.02 | | | | |
| TL00900 | 0.108 | 0.407 | 2.456 | YES | | YES | YES |
| TL00894 | 1.191 | 2.777 | 0.36 | YES | YES | YES | YES |
| TL00888 | 0.961 | 1.179 | 0.849 | | | | |
| TL00885 | 0.122 | 0.528 | 1.893 | YES | | YES | YES |
| TL00737 | 0.116 | 0.594 | 1.685 | YES | | YES | YES |
| TL00873 | 0.127 | 0.663 | 1.509 | YES | | YES | YES |
| TL00801 | 0.541 | 0.863 | 1.159 | | | | |
| TL00570 | 0.104 | 0.542 | 1.844 | YES | | YES | YES |
| TL00569 | 0.101 | 0.535 | 1.87 | YES | | YES | YES |
| TL00568 | 0.102 | 0.553 | 1.808 | YES | | YES | YES |
| TL00567 | 0.115 | 0.581 | 1.72 | YES | | YES | YES |
| TL00194 | 0.129 | 0.602 | 1.662 | YES | | YES | YES |
| TL00182 | 0.100 | 0.514 | 1.947 | YES | | YES | YES |
| TL00177 | 0.110 | 0.573 | 1.745 | YES | | YES | YES |
| TL00165 | 0.106 | 0.538 | 1.86 | YES | | YES | YES |
| TL00161 | 0.106 | 0.553 | 1.807 | YES | | YES | YES |
| TL00159 | 0.108 | 0.552 | 1.811 | YES | | YES | YES |
| TL00150 | 0.104 | 0.564 | 1.772 | YES | | YES | YES |
| TL00146 | 0.101 | 0.547 | 1.828 | YES | | YES | YES |
| TL00140 | 0.096 | 0.503 | 1.989 | YES | | YES | YES |
| TL00136 | 0.099 | 0.494 | 2.024 | YES | | YES | YES |
| TL00132 | 0.125 | 0.575 | 1.739 | YES | | YES | YES |
| TL00119 | 0.106 | 0.445 | 2.248 | YES | | YES | YES |
| TL00109 | 0.096 | 0.448 | 2.234 | YES | | YES | YES |
| TL00098 | 0.144 | 0.527 | 1.897 | YES | | YES | YES |
| TL00096 | 0.104 | 0.42 | 2.378 | YES | | YES | YES |
| TL00084 | 0.123 | 0.467 | 2.139 | YES | | YES | YES |
| TL00082 | 0.125 | 0.541 | 1.848 | YES | | YES | YES |
| TL00079 | 0.107 | 0.53 | 1.888 | YES | | YES | YES |
| TL00071 | 0.087 | 0.481 | 2.08 | YES | | YES | YES |
| TL00070 | 0.099 | 0.553 | 1.808 | YES | | YES | YES |
| TL00602 | 0.102 | 0.532 | 1.88 | YES | | YES | YES |
| TL00599 | 0.103 | 0.54 | 1.851 | YES | | YES | YES |
| TL00597 | 0.311 | 0.737 | 1.356 | | | | |
| TL00595 | 0.102 | 0.521 | 1.918 | YES | | YES | YES |
| TL00672 | 0.102 | 0.559 | 1.788 | YES | | YES | YES |
| TL00323 | 0.118 | 0.615 | 1.627 | YES | | YES | YES |
| TL00656 | 0.127 | 0.654 | 1.529 | YES | | YES | YES |
| TL00319 | 0.135 | 0.684 | 1.462 | | | | YES |
| TL00309 | 0.148 | 0.633 | 1.58 | YES | | YES | YES |
| TL00307 | 0.099 | 0.547 | 1.828 | YES | | YES | YES |
| TL00302 | 0.101 | 0.577 | 1.734 | YES | | YES | YES |
| TL00299 | 0.098 | 0.528 | 1.892 | YES | | YES | YES |
| TL00290 | 0.096 | 0.52 | 1.924 | YES | | YES | YES |
| TL00287 | 0.185 | 0.523 | 1.911 | YES | | YES | YES |
| TL00280 | 0.086 | 0.46 | 2.176 | YES | | YES | YES |
| TL00278 | 0.095 | 0.462 | 2.165 | YES | | YES | YES |
| TL00276 | 0.128 | 0.548 | 1.825 | YES | | YES | YES |
| TL00263 | 0.098 | 0.442 | 2.262 | YES | | YES | YES |
| TL00244 | 0.095 | 0.434 | 2.305 | YES | | YES | YES |
| TL00240 | 0.143 | 0.515 | 1.941 | YES | | YES | YES |
| TL00239 | 0.111 | 0.532 | 1.88 | YES | | YES | YES |
| TL00236 | 0.127 | 0.6 | 1.667 | YES | | YES | YES |
| TL00233 | 0.136 | 0.656 | 1.525 | YES | | YES | YES |
| TL00231 | 0.401 | 0.832 | 1.202 | | | | |
| TL00414 | 0.151 | 0.737 | 1.356 | | | | YES |
| TL00405 | 0.104 | 0.517 | 1.934 | YES | | YES | YES |
| TL00402 | 0.106 | 0.533 | 1.875 | YES | | YES | YES |
| TL00392 | 0.110 | 0.492 | 2.032 | YES | | YES | YES |
| TL00379 | 0.117 | 0.466 | 2.146 | YES | | YES | YES |
| TL00376 | 0.104 | 0.47 | 2.126 | YES | | YES | YES |
| TL00373 | 0.107 | 0.471 | 2.124 | YES | | YES | YES |
| TL00371 | 0.110 | 0.471 | 2.122 | YES | | YES | YES |
| TL00370 | 0.112 | 0.507 | 1.973 | YES | | YES | YES |
| TL00367 | 0.117 | 0.571 | 1.751 | YES | | YES | YES |
| TL00365 | 0.120 | 0.598 | 1.673 | YES | | YES | YES |
| TL00360 | 0.109 | 0.623 | 1.604 | YES | | YES | YES |
| TL00761 | 0.127 | 0.61 | 1.64 | YES | | YES | YES |
| TL00750 | 0.207 | 0.561 | 1.782 | YES | | YES | YES |
| TL00725 | 0.141 | 0.595 | 1.682 | YES | | YES | YES |
| TL00723 | 0.111 | 0.5 | 1.998 | YES | | YES | YES |
| TL00717 | 0.319 | 0.648 | 1.543 | YES | | YES | |
| TL00715 | 0.107 | 0.412 | 2.427 | YES | | YES | YES |
| TL00707 | 0.121 | 0.488 | 2.05 | YES | | YES | YES |
| TL00687 | 0.113 | 0.468 | 2.139 | YES | | YES | YES |
| TL01032 | 0.253 | 0.833 | 1.2 | | | | |
| TL01020 | 0.222 | 0.319 | 3.132 | YES | | YES | YES |
| TL00449 | 0.121 | 0.61 | 1.64 | YES | | YES | YES |
| TL00445 | 0.259 | 0.634 | 1.577 | YES | | YES | YES |
| TL00633 | 0.130 | 0.59 | 1.696 | YES | | YES | YES |
| TL00274 | 0.106 | 0.519 | 1.927 | YES | | YES | YES |
| TL00272 | 0.105 | 0.492 | 2.034 | YES | | YES | YES |
| TL01038 | 0.118 | 0.52 | 1.924 | YES | | YES | YES |
| TL01027 | 0.109 | 0.457 | 2.186 | YES | | YES | YES |
| TL01015 | 0.105 | 0.45 | 2.222 | YES | | YES | YES |
| TL00999 | 0.104 | 0.456 | 2.193 | YES | | YES | YES |
| TL00998 | 0.115 | 0.465 | 2.15 | YES | | YES | YES |
| TL00991 | 0.134 | 0.497 | 2.012 | YES | | YES | YES |
| TL00977 | 0.118 | 0.543 | 1.84 | YES | | YES | YES |
| TL00965 | 0.112 | 0.587 | 1.705 | YES | | YES | YES |
| TL00793 | 0.111 | 0.61 | 1.64 | YES | | YES | YES |
| TL00705 | 0.107 | 0.543 | 1.843 | YES | | YES | YES |
| TL00662 | 0.119 | 0.571 | 1.751 | YES | | YES | YES |
| TL00653 | 0.112 | 0.558 | 1.793 | YES | | YES | YES |
| TL00641 | 0.135 | 0.584 | 1.713 | YES | | YES | YES |
| TL00606 | 0.362 | 1.007 | 0.993 | | | | |
| TL00550 | 0.106 | 0.472 | 2.121 | YES | | YES | YES |
| TL00548 | 0.101 | 0.464 | 2.157 | YES | | YES | YES |
| TL00547 | 0.125 | 0.542 | 1.845 | YES | | YES | YES |
| TL00546 | 0.101 | 0.471 | 2.123 | YES | | YES | YES |
| TL00543 | 0.111 | 0.541 | 1.848 | YES | | YES | YES |
| TL00542 | 0.106 | 0.573 | 1.745 | YES | | YES | YES |
| TL00541 | 0.104 | 0.574 | 1.741 | YES | | YES | YES |
| TL00742 | 0.164 | 0.776 | 1.288 | | | | |
| TL00736 | 0.104 | 0.554 | 1.804 | YES | | YES | YES |
| TL00711 | 0.128 | 0.649 | 1.541 | YES | | YES | YES |
| TL00707 | 0.119 | 0.529 | 1.889 | YES | | YES | YES |
| TL00589 | 0.125 | 0.541 | 1.85 | YES | | YES | YES |
| TL00588 | 0.126 | 0.498 | 2.009 | YES | | YES | YES |
| TL00586 | 0.107 | 0.451 | 2.218 | YES | | YES | YES |
| TL00585 | 0.126 | 0.511 | 1.957 | YES | | YES | YES |
| TL00580 | 0.125 | 0.526 | 1.903 | YES | | YES | YES |
| TL00577 | 0.112 | 0.506 | 1.975 | YES | | YES | YES |
| TL00576 | 0.109 | 0.558 | 1.791 | YES | | YES | YES |
| TL00572 | 0.152 | 0.71 | 1.409 | | | | |
| | 1.001 | 1.001 | 1 | | | | |
| | 0.341 | 1.022 | 0.978 | | | | |
| TL00751 | 0.107 | 0.549 | 1.821 | YES | | YES | YES |
| TL00566 | 0.633 | 1.379 | 0.725 | | | | |
| TL00613 | 0.117 | 0.527 | 1.896 | YES | | YES | YES |
| TL00612 | 0.109 | 0.498 | 2.006 | YES | | YES | YES |
| TL00611 | 0.117 | 0.505 | 1.981 | YES | | YES | YES |
| TL00609 | 0.118 | 0.551 | 1.814 | YES | | YES | YES |
| TL00607 | 0.125 | 0.54 | 1.852 | YES | | YES | YES |
| TL00605 | 0.125 | 0.663 | 1.508 | YES | | YES | YES |
| TL00604 | 0.149 | 0.716 | 1.396 | | | | YES |

Result: According to Table 3, the marker data of the unknown microorganism was the same as the marker data of the lactic acid bacteria #73. The unknown microorganism was identified as the lactic acid bacteria #73.

While embodiments of the present invention have been illustrated and described, various modifications and improvements can be made by persons skilled in the art. The embodiments of the present invention are therefore described in an illustrative but not restrictive sense. It is intended that the present invention is not limited to the particular forms as illustrated, and that all the modifications not departing from the spirit and scope of the present invention are within the scope as defined in the appended claims.

1. A method for establishing a standardized detection system for identifying a microorganism, which comprises:
(i) providing a test cell line;
(ii) culturing the test cell line with and without the microorganism;
(iii) determining gene expression values in the test cell line cultured with and without the microorganism;
(iv) selecting a group of genes, whose gene expression values in the test cell line cultured without the microorganism is significantly different from that cultured with the microorganism as markers; and
(v) storing the marker data obtained as the standardized detection system.

2. The method according to claim 1, wherein the gene expression values are determined in a microarray.

3. The method according to claim 1, wherein the test cell line in step (i) is provided to show significantly different gene expression values of the test cell line cultured with and without the microorganism.

4. The method according to claim 1, wherein the gene expression values in step (iii) are determined by determining individual gene expression values of the test cell line.

5. The method according to claim 1, wherein the group of genes of step (iv) have an expression value ratio of the test cell line cultured with the microorganism to that cultured without the microorganism larger than 0.2.

6. The method according to claim 1, wherein the group of genes of step (iv) have an expression value ratio of the test cell line cultured with the microorganism to that cultured without the microorganism larger than 1.5.

7. The method according to claim 1, wherein the expression difference between the group of genes in step (iv) in the test cell line cultured with and without the microorganism is incorporated into the marker data.

8. The method according to claim 1, wherein more than one cell line is used in step (i); wherein the more than one cell line are derived from the same species.

9. A method for identifying an unknown microorganism as a standardized and recognized microorganism, which comprises:
(a) providing a standardized detection system for a standardized and recognized microorganism, wherein the detection system is prepared by the steps of:
(i) providing a test cell line;
(ii) culturing the test cell line with and without the standardized and recognized microorganism;
(iii) determining gene expression values in the test cell line cultured with and without the microorganism;
(iv) selecting a group of genes, whose gene expression values in the test cell line cultured without the microorganism is significantly different from that cultured with the microorganism as markers; and
(v) storing the marker data obtained as the standardized detection system;
(b) culturing the test cell line with and without the unknown microorganism;
(c) determining gene expression values in the test cell line cultured with and without the unknown microorganism;
(d) selecting a group of genes, whose expression in the test cell line cultured without the unknown microorganism is significantly different from that cultured with the unknown microorganism as markers;
(e) comparing the marker data obtained in step (d) with the marker data obtained in step (a), whereby the unknown microorganism is identified as the standardized and recognized microorganism if the marker data obtained in step (d) is similar to the marker data obtained in step (a) within an acceptable level.

10. The method according to claim 9, wherein the gene expression values are determined in a microarray.

11. The method according to claim 9, wherein the test cell line in step (i) of step (a) is provided to show significantly different gene expression values of the test cell line cultured with and without the standardized and recognized microorganism.

12. The method according to claim 9, wherein the gene expression values in step (iii) of step (a) and in step (c) are determined by determining individual gene expression values of the test cell line.

13. The method according to claim 9, wherein the group of genes in step (iv) of step (a) have an expression value ratio of the test cell line cultured with the standardized and recognized to that cultured without the standardized and recognized microorganism larger than 0.2.

14. The method according to claim 9, wherein the group of genes in step (iv) of step (a) have an expression value ratio of the test cell line cultured with the standardized and recognized microorganism to that cultured without the standardized and recognized microorganism larger than 1.5.

15. The method according to claim 9, wherein the expression difference between the group of genes in step (iv) in the test cell line cultured with and without the standardized and recognized microorganism is incorporated into the marker data.

16. The method according to claim 9, which is applied in quality control for manufacturing a microorganism composition.

17. The method according to claim 9, wherein more than one cell line is used in step (i); wherein the more than one cell line are derived from the same species.

18. A method for establishing a standardized detection system for detecting a specific function in a microorganism, which comprises:
(i) providing a test cell line;
(ii) culturing the test cell line with and without a reference microorganism having a specific function;
(iii) determining gene expression values in the test cell line cultured with and without the microorganism;
(iv) selecting a group of genes, whose gene expression values in the test cell line cultured without the microorganism is significantly different from that cultured with the microorganism as markers; and
(v) storing the marker data obtained as the standardized detection system.

19. The method according to claim 18, wherein the gene expression values are determined in a microarray.

20. The method according to claim 18, wherein the test cell line in step (i) is provided to show significantly different gene expression values of the test cell line cultured with and without the reference microorganism.

21. The method according to claim 18, wherein the gene expression values in step (iii) are determined by determining individual gene expression values related to the specific function of the test cell line.

22. The method according to claim 18, wherein the group of genes of step (iv) have an expression value ratio of the test cell line cultured with the reference microorganism to that cultured without the reference microorganism larger than 0.2.

23. The method according to claim 18, wherein the group of genes of step (iv) have an expression value ratio of the test cell line cultured with the reference microorganism to that cultured without the reference microorganism larger than 1.5.

24. The method according to claim 18, wherein the expression difference between the group of genes in step (iv) in the test cell line cultured with and without the reference microorganism is incorporated into the marker data.

25. The method according to claim 18, wherein more than one cell line is used in step (i); wherein the more than one cell line are derived from the same species.

26. A method for detecting a test microorganism having a specific function, which comprises:
(a) providing a standardized detection system for identifying the specific function in a reference microorganism, wherein the detection system is prepared by the steps of:
(i) providing a test cell line;
(ii) culturing the test cell line with and without the reference microorganism;
(iii) determining gene expression values in the test cell line cultured with and without the microorganism;
(iv) selecting a group of genes, whose gene expression values in the test cell line cultured without the microorganism is significantly different from that cultured with the microorganism as markers; and
(v) storing the marker data obtained as the standardized detection system;
(b) culturing the test cell line with and without the test microorganism;
(c) determining gene expression values related to the specific function of the test cell line cultured with and without the test microorganism;
(d) selecting a group of genes, whose expression in the test cell line cultured without the test microorganism is significantly different from that with the test microorganism as markers;
(e) comparing the marker data obtained in step (d) with the marker data obtained in step (a), whereby the test microorganism is identified to have the specific function if the marker data obtained in step (d) is similar to the maker data obtained in step (a) within an acceptable level.

27. The method according to claim 26, wherein the gene expression values are determined in a microarray.

28. The method according to claim 26, wherein the test cell line in step (i) of step (a) is provided to show significantly different gene expression values of the test cell line cultured with and without the reference microorganism.

29. The method according to claim 26, wherein the gene expression values in step (iii) of step (a) and in step (c) are determined by determining individual gene expression values related to the specific function of the test cell line.

30. The method according to claim 26, wherein the group of genes in step (iv) of step (a) have an expression value ratio of the test cell line cultured with the reference microorganism to that cultured without the reference microorganism larger than 0.2.

31. The method according to claim 26, wherein the group of genes in step (iv) of step (a) have an expression value ratio of the test cell line cultured with the reference microorganism to that cultured without the reference microorganism larger than 1.5.

32. The method according to claim 26, wherein the expression difference between the group of genes in step (iv) in the test cell line cultured with and without the reference microorganism is incorporated into the marker data.

33. The method according to claim 26, wherein more than one cell line is used in step (i); wherein the more than one cell line are derived from the same species.
